SEQUENCE LISTING (9 sequences)

SEQ ID NO: 1 - DNA, 21 bp, synthetic construct
  source 1..21: mol_type = other DNA
  misc_feature 1..21: note = Oligonucleotide
cgatctctctctctttctagt

SEQ ID NO: 2 - DNA, 21 bp, synthetic construct
  source 1..21: mol_type = other DNA
  misc_feature 1..21: note = Oligonucleotide
actagaaagagagagagatcg

SEQ ID NO: 3 - DNA, 15 bp, synthetic construct
  source 1..15: mol_type = other DNA
  misc_feature 1: note = psoralen-based compounds-linked nucleotide
tctttctctctctct

SEQ ID NO: 4 - DNA, 14 bp, synthetic construct
  source 1..14: mol_type = other DNA
  misc_feature 1..14: note = TFO (Hoogsteen base pair type)
  modified_base 4..7: mod_base = OTHER; note = protonated cytidine
  modified_base 11: mod_base = OTHER; note = protonated cytidine
tgtcccctttcttt

SEQ ID NO: 5 - DNA, 14 bp, synthetic construct
  source 1..14: mol_type = other DNA
  misc_feature 1..14: note = TFO (Reverse Hoogsteen base pair type)
tgtgtttggggtgt

SEQ ID NO: 6 - DNA, 16 bp, Homo sapiens
  source 1..16: mol_type = genomic DNA
cagaggggaaagagac

SEQ ID NO: 7 - DNA, 16 bp, Homo sapiens
  source 1..16: mol_type = genomic DNA
gtctctttcccctctg

SEQ ID NO: 8 - DNA, 15 bp, synthetic construct
  source 1..15: mol_type = other DNA
  modified_base 1..15: mod_base = OTHER; note = peptide nucleic acid
  misc_feature 1: note = psoralen-based compounds-linked nucleotide
tctttctctctctct

SEQ ID NO: 9 - DNA, 15 bp, synthetic construct
  source 1..15: mol_type = other DNA
  modified_base 1..15: mod_base = OTHER; note = peptide nucleic acid
  misc_feature 1: note = psoralen-based compounds-linked nucleotide
tctctctctctttct

(19) United States
(12) Patent Application Publication
(10) Pub. No.: US 2026/0216336 A1
(43) Pub. Date: Jul. 30, 2026

(54) NEW PSORALEN COMPOUND

(71) Applicant: NAGASAKI UNIVERSITY, Nagasaki-shi, Nagasaki (JP)

(72) Inventors: Asako YAMAYOSHI, Nagasaki (JP); Yu MIKAME, Nagasaki (JP); Juki NAKAO, Nagasaki (JP)

(73) Assignee: NAGASAKI UNIVERSITY, Nagasaki-shi, Nagasaki (JP)

(21) Appl. No.: 19/141,424

(22) PCT Filed: Dec. 22, 2023
(86) PCT No.: PCT/JP2023/046152

§ 371 Date: Jun. 20, 2025

(30) Foreign Application Priority Data
Dec. 22, 2022 (JP) 2022-206081
Jun. 28, 2023 (JP) 2023-106429

(51) Int. Cl. A61K 41/00; A61K 31/7088; C07D 405/14; C12N 15/10; C12N 15/113

(52) U.S. Cl. CPC A61K 41/0066; A61K 31/7088; C07D 405/14; C12N 15/10; C12N 15/113; C12N 2310/20

(57) ABSTRACT

The present invention provides a compound represented by the following formula (I):

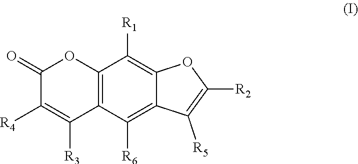

[each symbol in formula (I) is as described in the accompanying description], or a salt thereof.

### FIELD

The present invention relates to novel psoralen compounds and uses thereof.

### BACKGROUND

Psoralen and psoralen compounds are low molecular weight compounds used in PUVA therapy (phototherapy for skin diseases). These compounds are “intercalators” to be inserted between bases of a nucleic acid such as DNA or RNA, and it has been known that they form adducts with the nucleic acid when irradiated with light. By introducing psoralen into the 5′ end of a nucleic acid (antisense oligonucleotide, triplex-forming oligonucleotide, or the like) that forms base pairs with a target gene, site-specific formation of adducts with the target gene becomes possible (e.g., NPL 1). As a commercially available reagent for introducing psoralen into nucleic acids, the following compound is currently known.

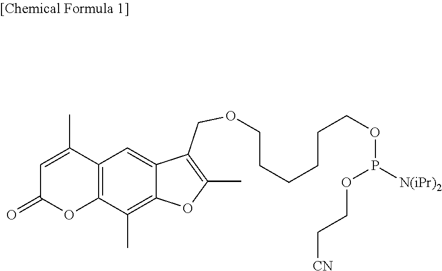

The above compound is Psoralen C6 Phosphoramidite (6-[4′-(Hydroxymethyl)-4,5′,8-trimethylpsoralen]-hexyl-1-O-(2-cyanoethyl)-(N,N-diisopropyl)-phosphoramidite) commercially available from GLEN RESEARCH, and is introduced into nucleic acids using the phosphoramidite method. Although Psoralen C6 Phosphoramidite exhibits favorable DNA crosslinking efficiency, it has problems in that the phosphoramidite method requires expensive and special synthesis equipment and synthesis of the compound consumes large amounts of reagents. Besides, since the synthesis using the phosphoramidite method involves water-prohibited reactions, consideration must be given to factors such as humidity and thus the experimental procedures are not easy. Another currently known psoralen compound is the following compound.

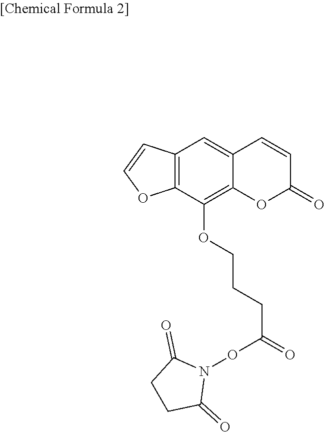 [SMILES: O=C(CCCOc1c2occc2cc2ccc(=O)oc12)ON1C(=O)CCC1=O]

The above compound is SPB (succinimidyl-[4-(psoralen-8-yloxy)]butyrate) commercially available from Thermo Fisher SCIENTIFIC, and is introduced into nucleic acids through a condensation reaction with a primary amine. SPB is advantages in that it can be introduced under standard labeling conditions using NHS esters, experimental procedures are easy, and the introduction site of SPB can be set freely depending on the introduction site of the primary amine.

By the way, in recent years, genome-editing technologies such as CRISPR (clustered regularly interspaced short palindromic repeats)/Cas9 system and ZFN is attracting attention. Genome-editing technologies are applied not only to modification of chromosomes in cells but also to methods for inactivating viral genes (NPLs 2 and 3). The most common method for inactivating viral genes using genome editing technologies is to use an “exogenous DNA cleaving enzyme”, and the action mechanism thereof is as follows: (A) the DNA cleaving enzyme is used to induce a double strand break (DSB) in genomic DNA; and (B) when the DSB is repaired through non-homologous end-joining, partial gene deletion is induced, thereby inactivating the viral gene (NPL 3). However, the above-described genome-editing technologies have the following problems: (1) it is necessary to introduce a DNA cleaving enzyme (such as Cas9 or TALEN) into cells while such an enzyme does not exist in eukaryotes; (2) genomic sequences that can be edited by these technologies are limited (for example, in the case of the CRISPR/Cas9 system, a DSB can be included only at a site several bases upstream from a PAM sequence (NGG)); (3) external control is difficult; and (4) in particular, technologies utilizing the CIRPSR/Cas9 system are difficult to commercialize since the patent rights for key technologies of the CIRPSR/Cas9 system is owned by a group in the United States.

### CITATION LIST

### Non Patent Literature

[NPL 1] Liu J., et al., J. Biol. Chem., 285 (3): 23198-23207 (2010)
[NPL 2] Doudna J. A., Charpentier E., Science., 346 (6213): 1258096 (2014)
[NPL 3] Tanaka A., et al., Leukemia., 27 (8): 1621-1627 (2013)

### SUMMARY

### Technical Problem

Studies conducted by the inventors of the present invention revealed that the DNA crosslinking efficiency of SPB is very low. With the foregoing in mind, it is an object of the present invention to provide a novel psoralen compound that has high DNA crosslinking efficiency and can be introduced into nucleic acids under standard labeling conditions using NHS esters. Also, it is another object of the present invention to provide a method for performing phototherapy or genome editing using such psoralen compounds.

### Solution to Problem

The inventors of the present invention came up with the idea that improving existing psoralen compounds might lead to compounds with improved DNA crosslinking efficiency. The inventors then focused their attention to SPB, which has a linker bound to the 8-carbon of the psoralen structure, and surprisingly found out that compounds adapted to have the above-described linker at another carbon atom of the psoralen structure, specifically at the 4′- or 5-carbon, exhibit markedly improved DNA crosslinking efficiency as compared to SPB. Based on these findings, the inventors further conducted in-depth studies, which led to completion of the present invention.

Specifically, the present invention provides the following.

[1]

A compound represented by the following formula (I) or a salt thereof:

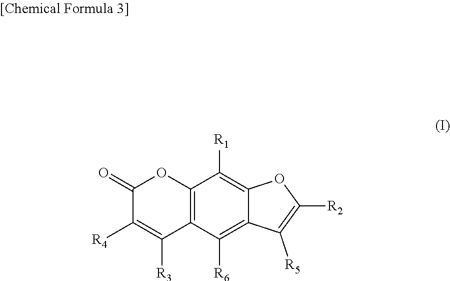

[in the formula (I):
R₁ is a hydrogen atom, an aliphatic group having 1 to 3 carbon atoms, or —O—R₇ [where R₇ is a hydrogen atom or an aliphatic group having 1 to 3 carbon atoms], or is represented by the following formula (II-1), (II-2), or (II-3):

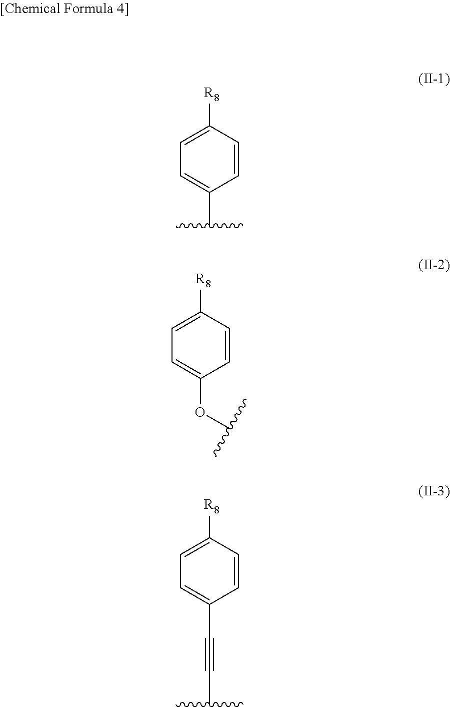

[in the formulae (II-1), (II-2), and (II-3), R₈ is a hydrogen atom, an aliphatic group having 1 to 3 carbon atoms, or —N(R₉)₂ or —O—R₉ [where R₉ is a hydrogen atom or an aliphatic group having 1 to 3 carbon atoms]],
R₂ is a hydrogen atom, an aliphatic group having 1 to 3 carbon atoms, or —O—R₁₀ [where R₁₀ is a hydrogen atom or an aliphatic group having 1 to 3 carbon atoms],
R₃ and R₄ are each independently a hydrogen atom, an aliphatic group having 1 to 3 carbon atoms, —O—R₁₁ [where R₁₁ is a hydrogen atom or an aliphatic group having 1 to 3 carbon atoms], or a phenyl group or naphthyl group in which each hydrogen atom may be substituted with an aliphatic group having 1 to 3 carbon atoms, or alternatively,
R₃ and R₄ together form a benzene ring or naphthyl ring in which each hydrogen atom may be substituted with an aliphatic group having 1 to 3 carbon atoms, and
one of R₅ and R₆ is a hydrogen atom, an aliphatic group having 1 to 3 carbon atoms, or —O—R₁₂ [where R₁₂ is a hydrogen atom or an aliphatic group having 1 to 3 carbon atoms] and the other one of R₅ and R₆ is represented by the following Formula (III-1) or (III-2):

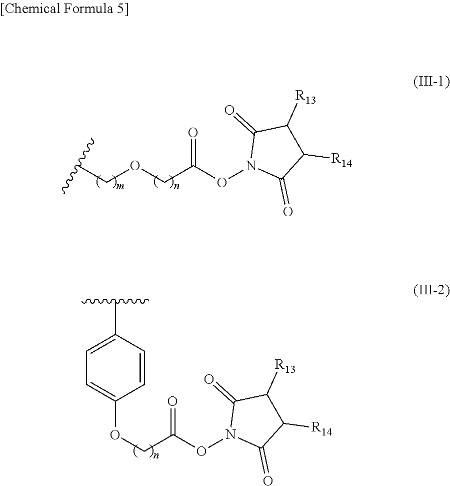

[in the formulae (III-1) and (III-2),
m is an integer of 0 to 4,
n is an integer of 1 to 5, and
R₁₃ and R₁₄ are each independently a hydrogen atom, an aliphatic group having 1 to 3 carbon atoms, or a polar group]].
   [2-1]

The compound or salt thereof according to [1], wherein R₁ is a hydrogen atom, an aliphatic group having 1 to 3 carbon atoms, or —O—R₇ [where R₇ is a hydrogen atom or an aliphatic group having 1 to 3 carbon atoms].

[2-2]

The compound or salt thereof according to [1] or [2-1], wherein R₁ to R₄ are each independently a hydrogen atom or an aliphatic group having 1 to 3 carbon atoms (preferably a methyl group).

[2-3]

The compound or salt thereof according to any one of [1] to [2-2], wherein R₁ to R₄ are all hydrogen atoms.

[2-4]

The compound or salt thereof according to any one of [1] to [2-2], wherein R₁ to R₃ are all methyl groups and R₄ is a hydrogen atom.

[3-1]

The compound or salt thereof according to any one of [1] to [2-4], wherein R₁₃ is a polar group (preferably a sulfo group).

[3-2]

The compound or salt thereof according to [1] or [3-1], wherein R₁₄ is a hydrogen atom or an aliphatic group having 1 to 3 carbon atoms (preferably a hydrogen atom).

[4-1]

The compound or salt thereof according to any one of [1] to [3-2], wherein m is 0 or 1.

[4-2]

The compound or salt thereof according to any one of [1] to [4-1], wherein n is 1.

[5]

The compound or salt thereof according to any one of [1] to [4-2], wherein R₆ is represented by the above formula (III-1) or (III-2).

[6]

The compound according to [1], represented by the following formula (IV-1) or (IV-2):

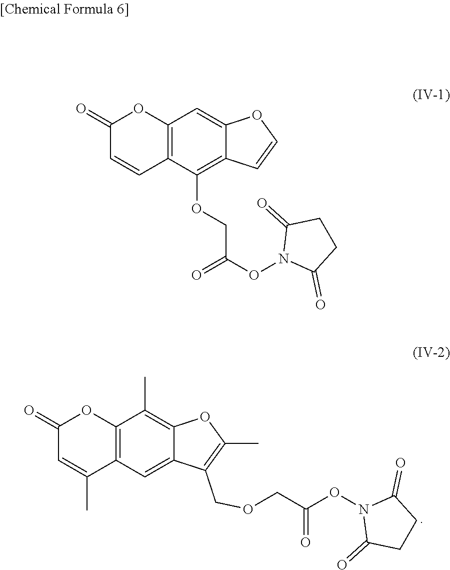 [SMILES: O=C(COc1c2ccoc2cc2oc(=O)ccc12)ON1C(=O)CCC1=O ; Cc1oc2c(C)c3oc(=O)cc(C)c3cc2c1COCC(=O)ON1C(=O)CCC1=O]

[7]

A nucleic acid having a structure represented by the following formula (V-1), (V-2), (V-3), or (V-4):

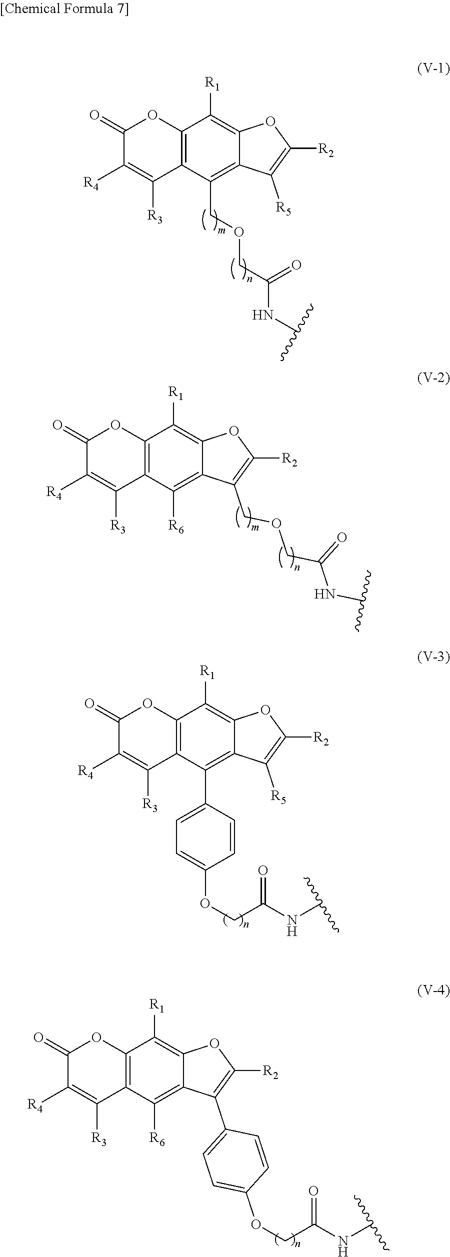

[in the formulae (V-1), (V-2), (V-3), and (V-4),
R₁ is independently a hydrogen atom, an aliphatic group having 1 to 3 carbon atoms, or —O—R₇ [where R₇ is a hydrogen atom or an aliphatic group having 1 to 3 carbon atoms], or is represented by the following formula (II-1), (11-2), or (11-3):

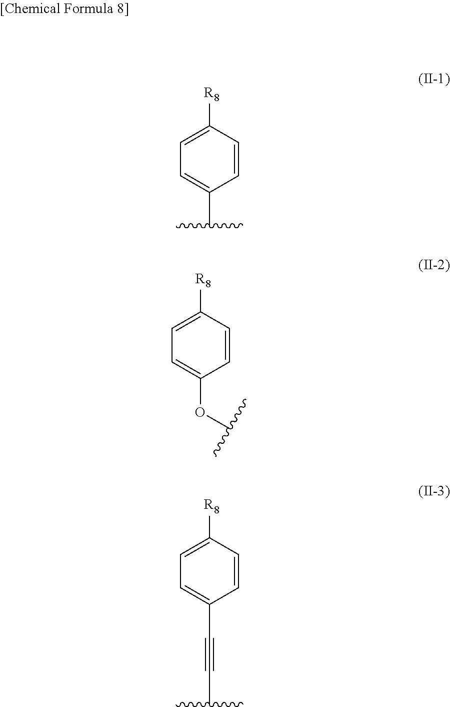

[in the formulae (II-1), (II-2), and (II-3), R₈ is a hydrogen atom, an aliphatic group having 1 to 3 carbon atoms, or —N(R₉)₂ or —O—R₉ [where R₉ is a hydrogen atom or an aliphatic group having 1 to 3 carbon atoms]],
R₂ is a hydrogen atom, an aliphatic group having 1 to 3 carbon atoms, or —O—R₁₀ [where R₁₀ is a hydrogen atom or an aliphatic group having 1 to 3 carbon atoms],
R₃ and R₄ are each independently a hydrogen atom, an aliphatic group having 1 to 3 carbon atoms, —O—Ru [where Ru is a hydrogen atom or an aliphatic group having 1 to 3 carbon atoms], or a phenyl group or naphthyl group in which each hydrogen atom may be substituted with an aliphatic group having 1 to 3 carbon atoms, or alternatively
R₃ and R₄ together form a benzene ring or naphthyl ring in which each hydrogen atom may be substituted with an aliphatic group having 1 to 3 carbon atoms,
R₅ and R₆ are each independently a hydrogen atom, an aliphatic group having 1 to 3 carbon atoms, or —O—R₁₂ [where R₁₂ is a hydrogen atom or an aliphatic group having 1 to 3 carbon atoms],
m is an integer of 0 to 4, and
n is an integer of 1 to 5].
   [8-1]

The nucleic acid according to [7], wherein R₁ is a hydrogen atom, an aliphatic group having 1 to 3 carbon atoms, or —O—R₇ [where R₇ is a hydrogen atom or an aliphatic group having 1 to 3 carbon atoms].

[8-2]

The nucleic acid according to [7] or [8-1], wherein R₁ to R₄ are each independently a hydrogen atom or an aliphatic group having 1 to 3 carbon atoms (preferably a methyl group).

[8-3]

The nucleic acid according to any one of [7] to [8-2], wherein R₁ to R₄ are all hydrogen atoms.

[8-4]

The nucleic acid according to any one of [7] to [8-2], wherein R₁ to R₃ are all methyl groups and R₄ is a hydrogen atom.

[8-5]

The nucleic acid according to any one of [7] to [8-4], wherein m is 0 or 1.

[8-6]

The nucleic acid according to any one of [7] to [8-5], wherein n is 1.

[9]

The nucleic acid according to [7], having a structure represented by the following formula (VI-1) or (VI-2):

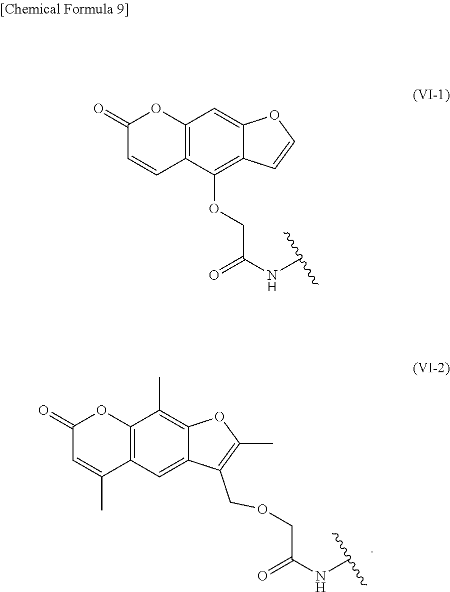 [SMILES: *NC(=O)COc1c2ccoc2cc2oc(=O)ccc12 ; *NC(=O)COCc1c(C)oc2c(C)c3oc(=O)cc(C)c3cc12]

[10]

The nucleic acid according to any one of [7] to [9], which is a triplex-forming oligonucleotide or an antisense oligonucleotide.

[11]

The nucleic acid according to any one of [7] to [10], wherein the triplex-forming oligonucleotide is DNA or PNA.

[12]

A method for producing the nucleic acid according to any one of [7] to [11], comprising a step of reacting the compound or salt thereof according to any one of [1] to [6] with a nucleic acid having an amino group.

[13-1]

A phototherapeutic agent, comprising the compound or salt thereof according to any one of [1] to [6] or the nucleic acid according to any one of [7] to [11].

[13-2]

The agent according to [13-1] for treating skin diseases.

[13-3]

The agent according to [13-1] for treating cancer.

[14]

A method for producing cells having modified double-stranded DNA, comprising
(1) a step of contacting double-stranded DNA including a target sequence and present in cells with the nucleic acid according to any one of [7] to [11] including a sequence complementary to the target sequence, and
(2) a step of irradiating the cells with light.
   [15-1]

The method according to [14], wherein the target sequence is derived from a virus.

[15-2]

The method according to [15-1], wherein the virus is a retrovirus.

[16-1]

A method for phototherapeutic treatment of a mammal, comprising a step of administering an effective amount of the compound or salt thereof according to any one of [1] to [6] or the nucleic acid according to any one of [7] to [11] to the mammal, and a step of irradiating the skin of the mammal with light.

[16-2]

The method according to [16-1] for treating skin diseases.

[16-3]

The method according to [16-1] for treating cancer.

[17-1]

The compound or salt thereof according to any one of [1] to [6] or the nucleic acid according to any one of [7] to [11] for use in the phototherapy.

[17-2]

The compound or salt thereof according to any one of [1] to [6] or the nucleic acid according to any one of [7] to [11] for use in the treatment of skin diseases.

[17-3]

The compound or salt thereof according to any one of [1] to [6] or the nucleic acid according to any one of [7] to [11] for use in the treatment of cancer.

[18-1]

Use of the compound or salt thereof according to any one of [1] to [6] or the nucleic acid according to any one of [7] to [11] in the manufacture of a phototherapeutic agent.

[18-2]

The use according to [18-1], wherein the phototherapeutic agent is a therapeutic agent for skin diseases.

[18-3]

The use according to [18-1], wherein the phototherapeutic agent is a therapeutic agent for cancer.

[19-1]

An agent for modifying double-stranded DNA, comprising the nucleic acid according to any one of [7] to [11].

[19-2]

The agent according to [19-1], wherein the agent for modifying is a therapeutic agent for viral infections.

### Advantageous Effects of Invention

The novel psoralen compounds of the present invention have high DNA crosslinking efficiency and are superior in terms of handleability. The present invention thus enables highly effective PUVA therapy, as well as genome editing with high editorial efficiency.

### BRIEF DESCRIPTION OF DRAWINGS

FIG. 1 (a) Sequences of the target-duplex (ds-DNA) and TFO. The sequences in (a) are designated as SEQ ID NOs: 1 to 3 from top to bottom, respectively. Analysis of the photo-crosslinking reaction of Ps-TFO 16 with target-duplex (b), Ps-TFO 17 with target-duplex (d) and Ps-TFO 18 with target-duplex (f) was performed using denaturing PAGE (15% polyacrylamide/7 M urea 80% formamide/TBE). Quantification of photo-crosslinking efficiency of Ps-TFO 16 (c) and Ps-TFO 17 (e) was conducted by measuring TAMRA fluorescence. The polyacrylamide gel of Ps-TFO 18 was stained with SYBR™ Gold.

FIG. 2 (a) Sequences and structures of the target ds-DNA and TFO (Ps-PNA). The sequence of Ps-PNA (from the N-terminus to the C-terminus) is designated as SEQ ID NO: 8. (b, c) Analysis of the photo-crosslinking reaction of the strand invasion of mixtures of S linker Trioxsalen-PNA with the target ds-DNA (Py-DNA/Pu-DNA) was performed using denaturing PAGE (15% polyacrylamide/7 M urea/20% formamide/TBE). [Py-DNA]=[Pu-DNA]=[S linker Trioxsalen-PNA]=1.0 M, 10 mM phosphate buffer (pH 5.3), 200 mM NaCl, and 0.1 mM EDTA. The photo-crosslinking efficiency was quantified by measuring the fluorescence of TAMRA bound to the 3′ end of Pu-DNA.

FIG. 3 (a) Sequences and structures of the target ds-DNA and TFO (Ps-PNA). (b) Analysis of the photo-crosslinking reaction of mixtures of O linker Trioxsalen-PNA with the target ds-DNA (Py-DNA/Pu-DNA) was performed using denaturing PAGE (15% polyacrylamide/7 M urea/20% formamide/TBE). [Py-DNA]=[Pu-DNA]=[O linker Trioxsalen-PNA]=1.0 M, 10 mM phosphate buffer (pH 5.3), 200 mM NaCl, and 0.1 mM EDTA. The photo-crosslinking efficiency was quantified by measuring the fluorescence of TAMRA bound to the 3′ end of Pu-DNA.

FIG. 4 (a) DNA structures: S Linker Trioxsalen-PNA and O Linker Trioxsalen-PNA. (b) The result of comparing the DNA crosslinking efficiencies of S Linker Trioxsalen-PNA and O Linker Trioxsalen-PNA (n=3 for each).

FIG. 5 (Upper part) The sequence and structure of Trioxsalen-tc-PNA targeting a hypothetical sequence (sequence that has been confirmed to be capable of stable triplex formation: Nakao et al., ChemBioChem, e202200789, 2023) (note here that there are two mismatched base pairs between Trioxsalen-tc-PNA and the target sequence). The 3×8-amino-3,6-dioxaoctanoic acid linker is used not only to cause a PNA sequence located on the N-terminal side of the linker to form a triplex with the target double-stranded DNA, but also, after the triplex has been formed, to induce base pairing by causing invasion of a PNA sequence located on the C-terminal side of the linker into the target double-stranded DNA. Since the PNA carries no charge, RRR is introduced to the C-terminus in order to improve the water solubility of the PNA and interactions of the PNA with nucleic acids (negatively charged). (Lower part) Information regarding a plasmid vector containing the target hypothetical sequence.

FIG. 6 Results of evaluating the intracellular activity of Trioxsalen-tc-PNA shown in FIG. 5 (n=3 for each; error bars: standard deviation). When the target sequence in the plasmid vector is cleaved by Trioxsalen-tc-PNA and UV irradiation, the firefly luciferase activity decreases and the Renilla luciferase activity increases (on the right in FIG. 6).

FIG. 7 shows a sequence corresponding to the sequence shown in FIG. 5 with the mismatched base pairs therein corrected. In the target of TFO (tc-PNA), the sequence (from the N-terminus to the C-terminus) located on the N-terminal side of the 3×8-amino-3,6-dioxaoctanoic acid linker is designated as SEQ ID NO: 9.

### DESCRIPTION OF EMBODIMENTS

### 1. Psoralen Compound of the Present Invention

The present invention provides a novel psoralen compound that can be used as an intercalator, as with psoralen. Specifically, the present invention provides a compound represented by the following formula (I) or a salt thereof:

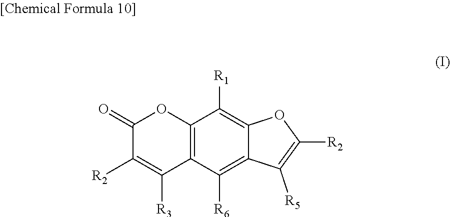

[in the formula (I):
R₁ is a hydrogen atom, an aliphatic group having 1 to 3 carbon atoms, or —O—R₇ [where R₇ is a hydrogen atom or an aliphatic group having 1 to 3 carbon atoms], or is represented by the following formula (II-1), (II-2), or (II-3):

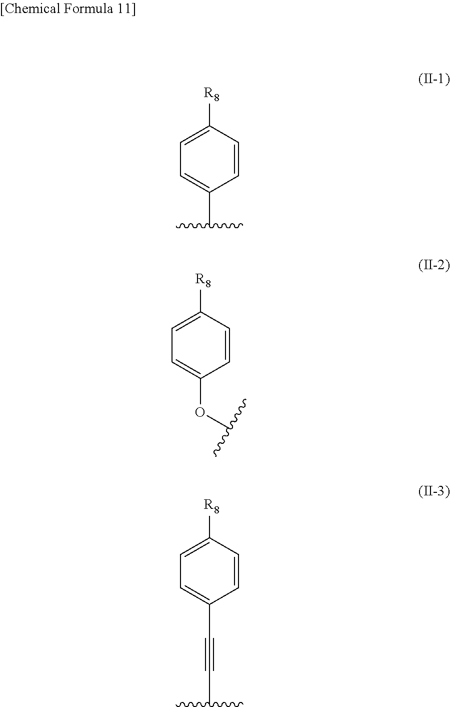

[in the formulae (II-1), (II-2), and (II-3), R₈ is a hydrogen atom, an aliphatic group having 1 to 3 carbon atoms, or —N(R₉)₂ or —O—R₉ [where R₉ is a hydrogen atom or an aliphatic group having 1 to 3 carbon atoms]],
R₂ is a hydrogen atom, an aliphatic group having 1 to 3 carbon atoms, or —O—R₁₀ [where R₁₀ is a hydrogen atom or an aliphatic group having 1 to 3 carbon atoms],
R₃ and R₄ are each independently a hydrogen atom, an aliphatic group having 1 to 3 carbon atoms, —O—R₁₁ [where R₁₁ is a hydrogen atom or an aliphatic group having 1 to 3 carbon atoms], or a phenyl group or naphthyl group in which each hydrogen atom may be substituted with an aliphatic group having 1 to 3 carbon atoms, or alternatively, R₃ and R₄ together form a benzene ring or naphthyl ring in which each hydrogen atom may be substituted with an aliphatic group having 1 to 3 carbon atoms, and
one of R₅ and R₆ is a hydrogen atom, an aliphatic group having 1 to 3 carbon atoms, or —O—R₁₂ [where R₁₂ is a hydrogen atom or an aliphatic group having 1 to 3 carbon atoms] and the other one of R₅ and R₆ is represented by the following Formula (III-1) or (III-2):

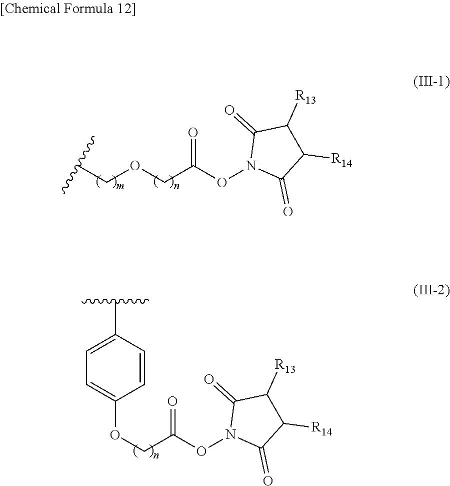

[in the formulae (III-1) and (III-2),
m is an integer of 0 to 4,
n is an integer of 1 to 5, and
R₁₃ and R₁₄ are each independently a hydrogen atom, an aliphatic group having 1 to 3 carbon atoms, or a polar group]]
   (hereinafter, this compound may be referred to as “the compound of the present invention”).

In the formulae (III-1) and (III-2), the ether bond may be substituted with a thioether bond. Accordingly, in the present specification, the above formulae (III-1) and (III-2) are interchangeable with the following formulae (III′-1) and (III′-2), respectively.

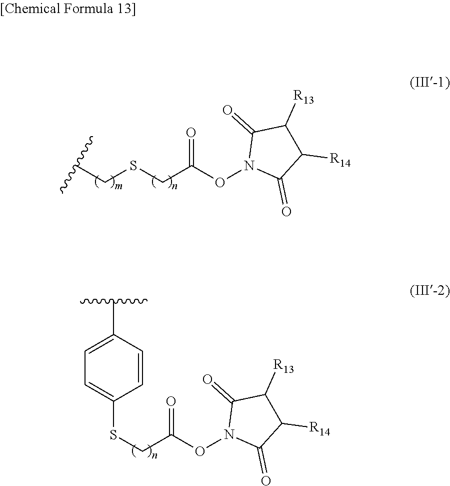

In one embodiment, the compound of the present invention is one in which R₁ is a hydrogen atom, an aliphatic group having 1 to 3 carbon atoms, or —O—R₇ [where R₇ is a hydrogen atom or an aliphatic group having 1 to 3 carbon atoms]. In particular, preferred is one in which R₁ to R₄ in the above formula (I) are each independently a hydrogen atom or an aliphatic group having 1 to 3 carbon atoms (preferably a methyl group) or a salt thereof, and more preferred is one in which R₁ to R₄ are all hydrogen atoms or a salt thereof, or one in which R₁ to R₃ are all methyl groups and R₄ is a hydrogen atom or a salt thereof.

In the case where the compound of the present invention is one in which R₁ is represented by the above formula (II-1), (II-2) or (II-3) or a salt thereof, R₈ is preferably a hydrogen atom, —N(R₉)₂, or —O—R₉ [where R₉ is a hydrogen atom or an aliphatic group having 1 to 3 carbon atoms (preferably a methyl group)].

In one embodiment, the compound of the present invention is one in which one of R₅ and R₆ in the formula (I) is a hydrogen atom or an aliphatic group having 1 to 3 carbon atoms (preferably a methyl group) and is particularly preferably a hydrogen atom. Also preferred is one in which R₈ in the formula (I) is a hydrogen atom, an aliphatic group having 1 to 3 carbon atoms, or —O—R₁₂ [where R₁₂ is a hydrogen atom or an aliphatic group having 1 to 3 carbon atoms](i.e., R₆ in the formula (I) is represented by the formula (III-1) or (III-2)) or a salt thereof.

The polar group in the above formulae (III-1) and (III-2) is not limited to a particular polar group as long as it improves the water solubility of the compound of the present invention, and may be, for example, an amino group, a carboxyl group, a hydroxyl group, or a sulfo group, and is preferably a sulfo group. In one embodiment, the compound of the present invention is one in which R₁₃ in the above formula (III-1) or (III-2) is a polar group (preferably a sulfo group) and R₁₄ is a hydrogen atom or an aliphatic group having 1 to 3 carbon atoms (preferably a hydrogen atom).

In one embodiment, the compound of the present invention is one in which m in the above formula (III-1) or (III-2) is 0 or 1. In another embodiment, the compound of the present invention is one in which n in the above formula (III-1) or (III-2) is 1. Also preferred is one in which m is 0 or 1 and n is 1 in the above formula (III-1) or a salt thereof.

More specific examples of the compound of the present invention include compounds represented by the following formulae (I-1) to (I-24) and salts thereof. The formula (I-13) corresponds to the compound in which R₃ and R₄ in the formula (I) together form a benzene ring, and the formula (I-14) corresponds to the compound in which R₃ and R₄ in the formula (I) together form a naphthyl ring.

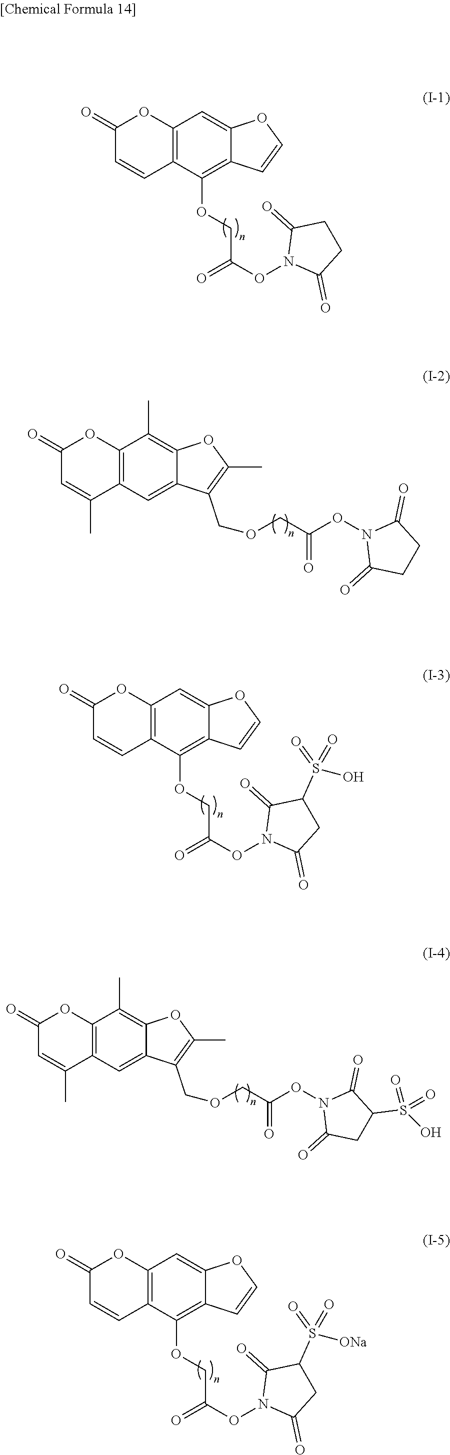

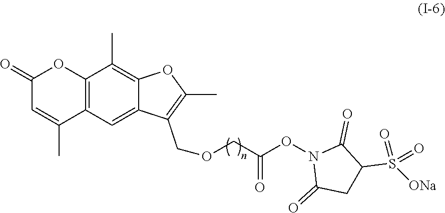

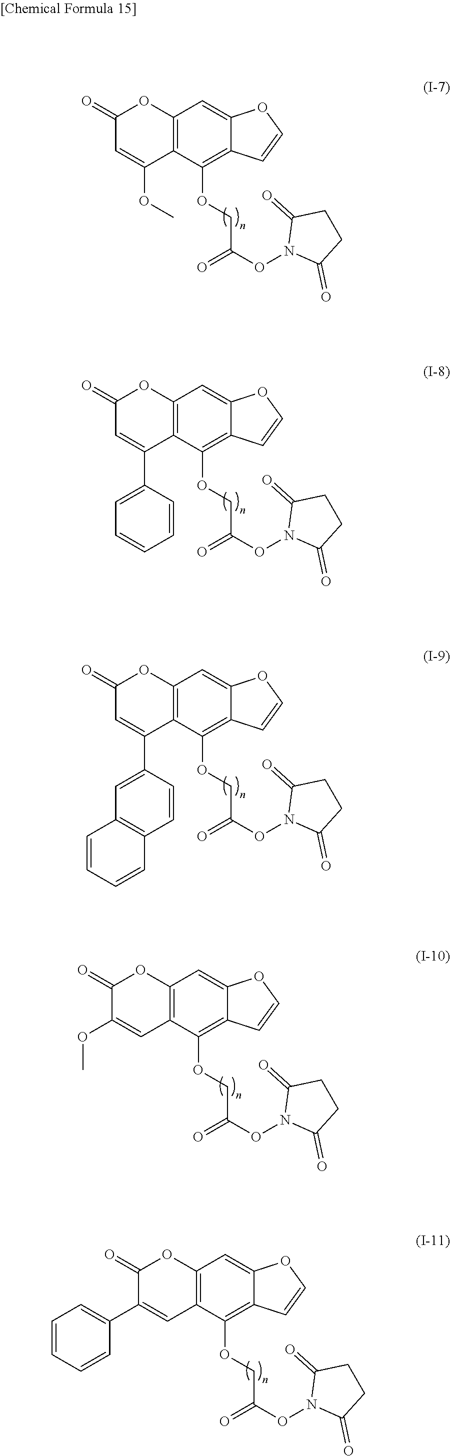

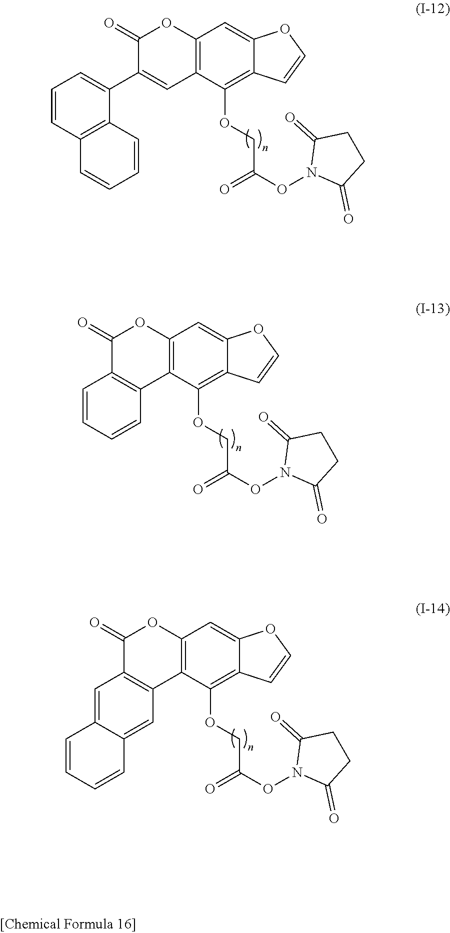

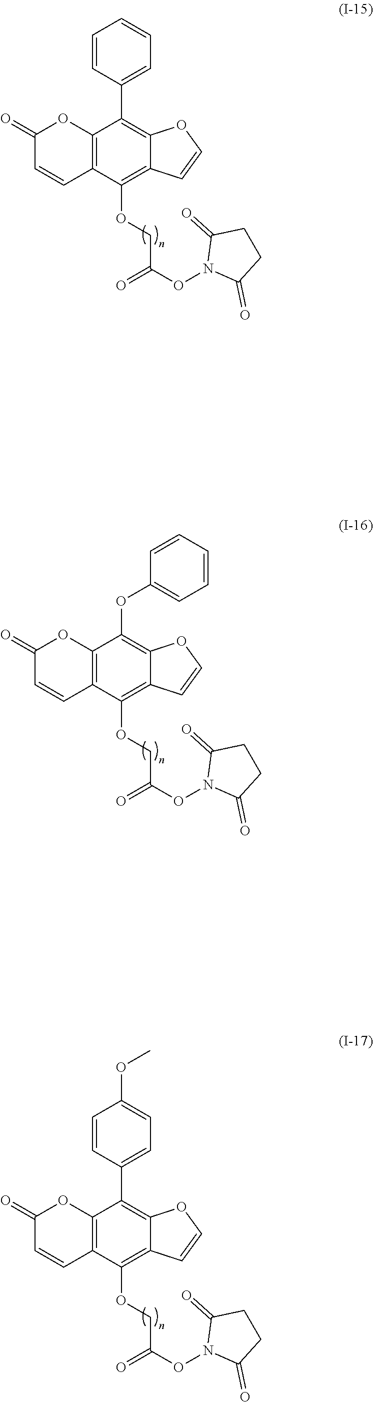

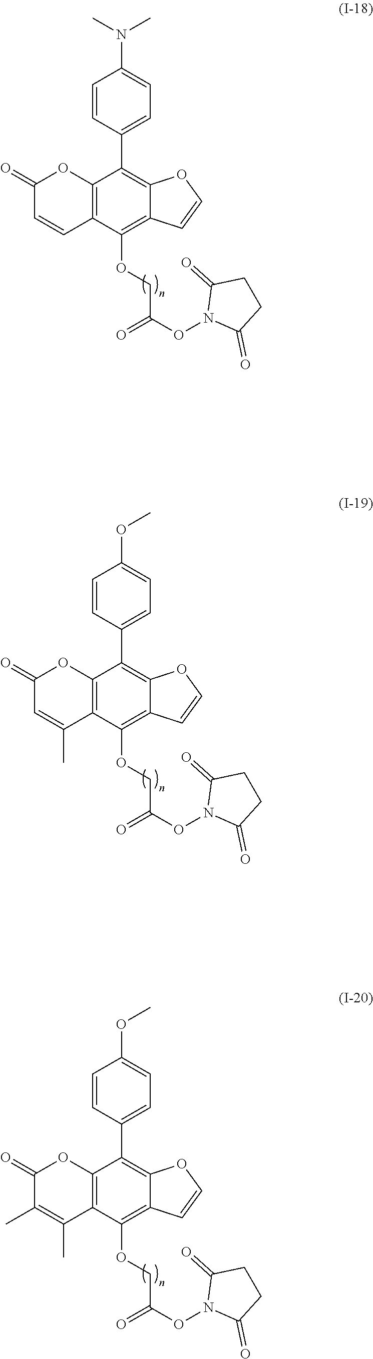

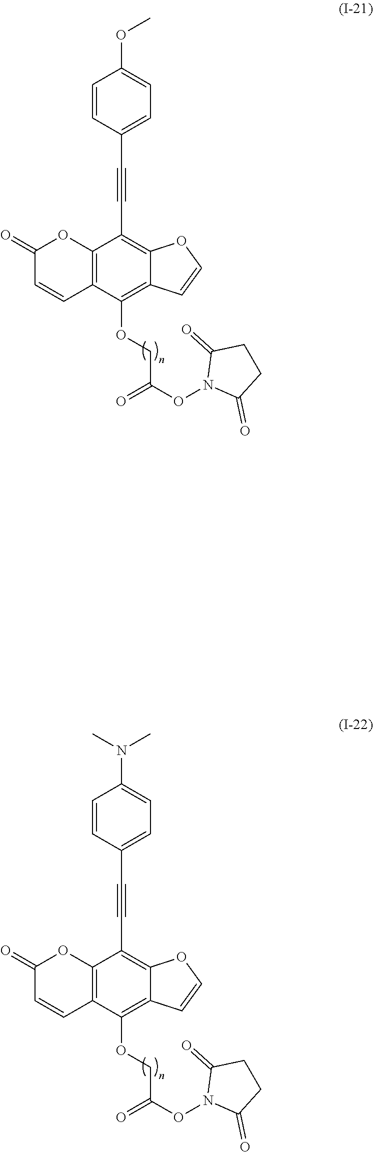

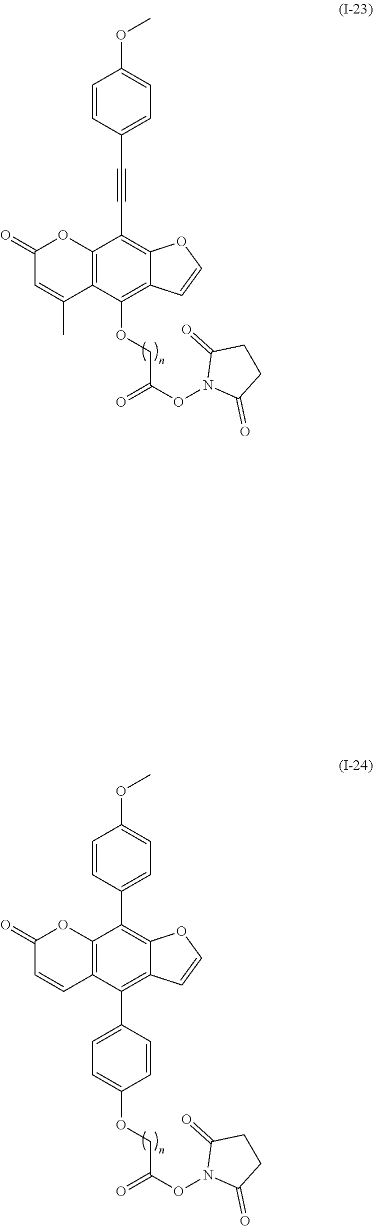

In the above formulae (I-1) to (I-24), n is an integer of 1 to 5 (preferably 1).

Of the compounds represented by the above formulae (I-1) to (I-24), particularly preferred is the compound represented by the following formula (IV-1) or (IV-2).

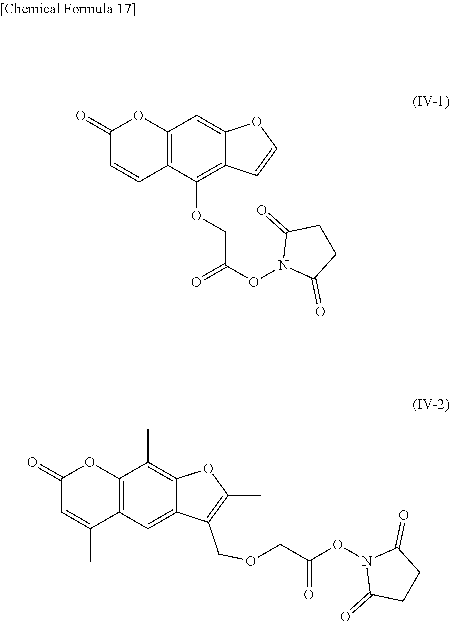 [SMILES: O=C(COc1c2ccoc2cc2oc(=O)ccc12)ON1C(=O)CCC1=O ; Cc1oc2c(C)c3oc(=O)cc(C)c3cc2c1COCC(=O)ON1C(=O)CCC1=O]

The salt of the compound represented by the above formula (I) varies depending on the type of the compound, and examples thereof include: base addition salts including inorganic base salts such as alkali metal salts (such as sodium salts and potassium salts), alkaline-earth metal salts (such as calcium salts and magnesium salts), aluminum salts, and ammonium salts, and organic base salts such as trimethylamine, triethylamine, pyridine, picoline, ethanolamine, diethanolamine, triethanolamine, dicyclohexylamine, and N,N′-dibenzylethylenediamine; and acid addition salts including inorganic acid salts such as hydrochloride, hydrobromide, sulfate, hydroiodide, nitrate, and phosphate, and organic acid salts such as citrate, oxalate, acetate, formate, propionate, benzoate, trifluoroacetate, maleate, tartrate, methanesulfonate, benzenesulfonate, and para-toluenesulfonate.

In the case where the compound represented by the above formula (I) or a salt thereof has isomers such as optical isomers, stereoisomers, regioisomers, and rotamers, any of the isomers and mixtures thereof are also encompassed in the compound of the present invention. Each of these isomers can be obtained as a single product by a method known per se, such as a synthesis method, a separation method (e.g., concentration, solvent extraction, column chromatography, or recrystallization), or an optical resolution method (e.g., fractional crystallization, a chiral column method, or a diastereomer method).

The compound represented by the above formula (I) or a salt thereof may be in the form of crystals, and regardless of whether it has only one crystal form or a mixture of crystal forms, it is encompassed in the compound of the present invention. Crystals can be produced by causing crystallization using a crystallization method known per se.

The compound represented by the above formula (I) or a salt thereof may be a solvate (e.g., a hydrate) or an ansolvate (e.g., a non-hydrate), and both of them are encompassed in the compound of the present invention.

The compound of the present invention encompasses those labeled with an isotope (e.g., ³H, ¹⁴C, ³⁵S, or ¹²⁵I) or the like.

As used herein, the term “aliphatic group” refers to a straight or branched hydrocarbon chain that is fully saturated or includes one or more unsaturated bonds. The aliphatic group having 1 to 3 carbon atoms may be, for example, a straight or branched alkyl group (e.g., a methyl group, ethyl group, propyl groups, or isopropyl group), an alkenyl group (e.g., a vinyl group, allyl group, or 2-propynyl group), or an alkynyl group (e.g., an ethynyl group, 1-propynyl group, or 2-propynyl group). Examples of the naphthyl group include a 1-naphthyl group and a 2-naphthyl group.

The compound of the present invention can be produced by a method known per se. For example, it can be produced by the following steps.

### Scheme 1, Steps a to d: The Synthesis of Compounds ii to v

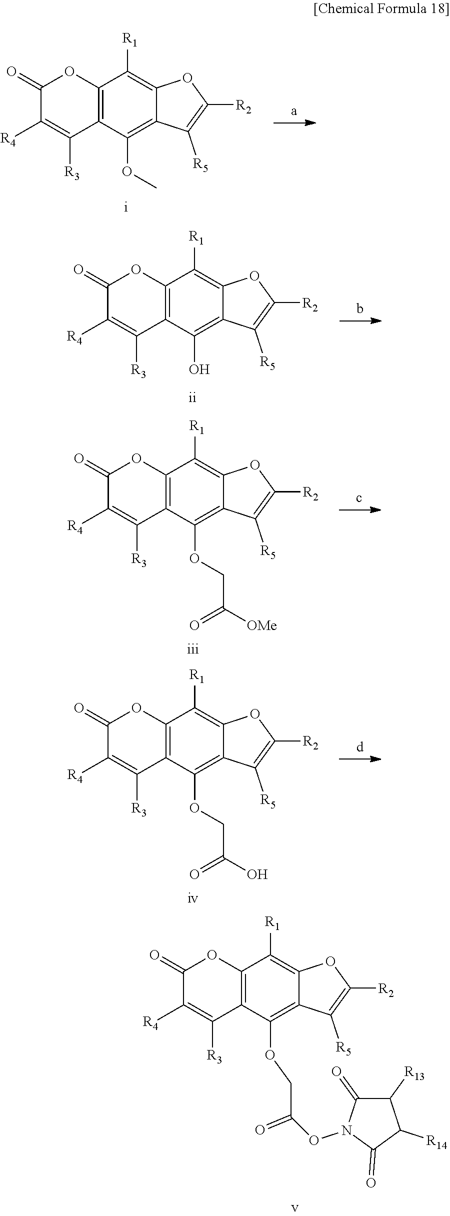

[In the above formulae, the definitions of R₁ to R₈ are the same as the definitions of those in the above formula (I), and the definitions of R₁₃ and R₁₄ are the same as the definitions of those in the above formula (III-1).]

Compound i may be a commercially available product (e.g., 5-methoxypsoralen) or may be synthesized by a method known per se. The step a can be performed according to the procedure reported in E. C. Row, S. A. Brown, A. V. Stachulski, M. S. Lennard, Org. Biomol. Chem. 2006, 4, 1604-1610.

The step b can be performed, for example, according to the following procedure. To a DMF solution containing compound ii and methyl bromoacetate is added K₂CO₃ at room temperature. The mixture is stirred overnight at 80° C. under an N₂ atmosphere, then water is added thereto. The whole is extracted with EtOAc twice, and the combined organic layers are dried over Na₂SO₄ and concentrated in vacuo. The residue is dissolved in hexane/EtOAc=3/1, and the resulting precipitate is collected by filtration. The solid is washed with hexane/EtOAc=3/1 and dried in vacuo to give compound iii.

The step c can be performed, for example, according to the following procedure. To MeOH/THF=1/1 solution containing the compound iii is added 1 M NaOH aq at room temperature. The mixture is stirred for 1 h, then the mixture is acidified with 5N HCl aq. The whole is extracted with EtOAc five times, and the combined organic layers are dried over Na₂SO₄ and concentrated in vacuo to give compound iv.

The step d can be performed, for example, according to the following procedure. To a DMF solution containing the compound iv and N-hydroxysuccinimide is added 1-ethyl-3-(3-dimethylaminopropyl)carbodiimide hydrochloride at room temperature. The mixture is stirred overnight at the same temperature under an N₂ atmosphere, then water is added and the whole is extracted with CH₂Cl₂ three times. The combined organic layers are washed with water twice, dried over Na₂SO₄, and concentrated in vacuo to give compound v.

### Scheme 2, Steps a to d: The Synthesis of Compounds vii to x

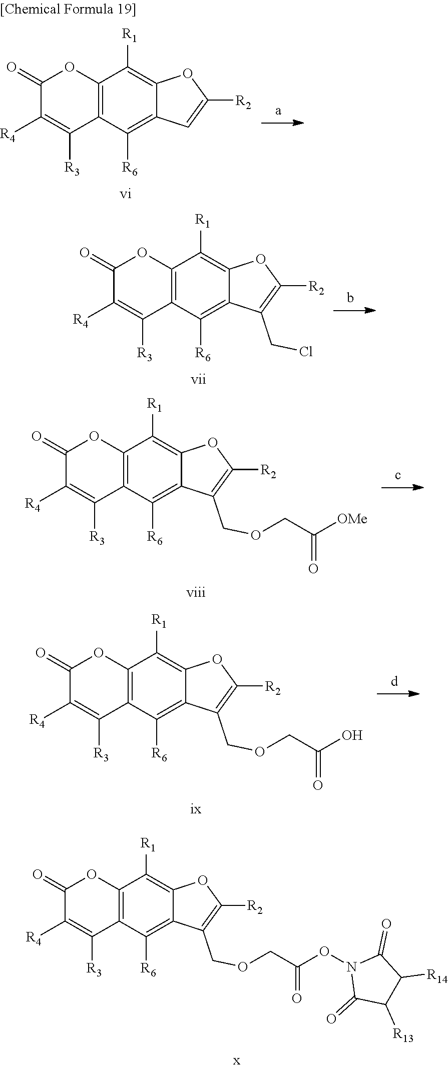

[In the above formulae, the definitions of R₁ to R₄ and R₆ are the same as the definitions of those in the above formula (I), and the definitions of R₁₃ and R₁₄ are the same as the definitions of those in the above formula (III-1).]

Compound vi may be a commercially available product (e.g., trioxysalen) or may be synthesized by a method known per se. The step a can be performed, for example, according to the procedure reported in A. Mukherjee, M. K. Vasquez, Nucleic Acids Res. 2016, 44, 1151-1160.

The step b can be performed, for example, according to the following procedure. A methyl glycolate solution containing compound vii is heated at 100° C. The mixture is stirred for 5 h at the same temperature under an N₂ atmosphere, then water is added and the whole is extracted with EtOAc three times. The combined organic layers are washed with water twice, dried over Na₂SO₄, and concentrated in vacuo. The residue is purified by silica gel column chromatography (hexane/EtOAc=40:1 to 1:1) to give compound viii.

The step c can be performed, for example, according to the following procedure. To an MeOH/THF=1/1 solution containing the compound viii is added 1 M NaOH aq at room temperature. The mixture is stirred for 3.5 h, then the mixture is acidified with 5N HCl aq. The whole is extracted with EtOAc five times, and the combined organic layers are dried over Na₂SO₄ and concentrated in vacuo to give compound ix.

The step d can be performed, for example, according to the following procedure. To a DMF solution containing the compound xi and N-hydroxysuccinimide is added 1-ethyl-3-(3-dimethylaminopropyl)carbodiimide hydrochloride at room temperature. The mixture is stirred overnight at the same temperature under an N₂ atmosphere, then distilled water is added and the whole is extracted with CH₂Cl₂ three times. The combined organic layers are washed with water twice, dried over Na₂SO₄, and concentrated in vacuo to give compound x.

### 2. Nucleic Acid of the Present Invention

The compound of the present invention has an N-hydroxysuccinimide (NHS) structure. Accordingly, through a condensation reaction between the NHS structure of the compound of the present invention and a primary amine as a compound of interest, the compound of the present invention can be easily introduced into the compound of interest. The compound of interest is not limited to a particular compound as long as it has an amino group, and examples thereof include nucleic acids. Therefore, viewed from another aspect, the present invention provides a nucleic acid having a structure represented by the following formula (V-1), (V-2), (V-3), or (V-4):

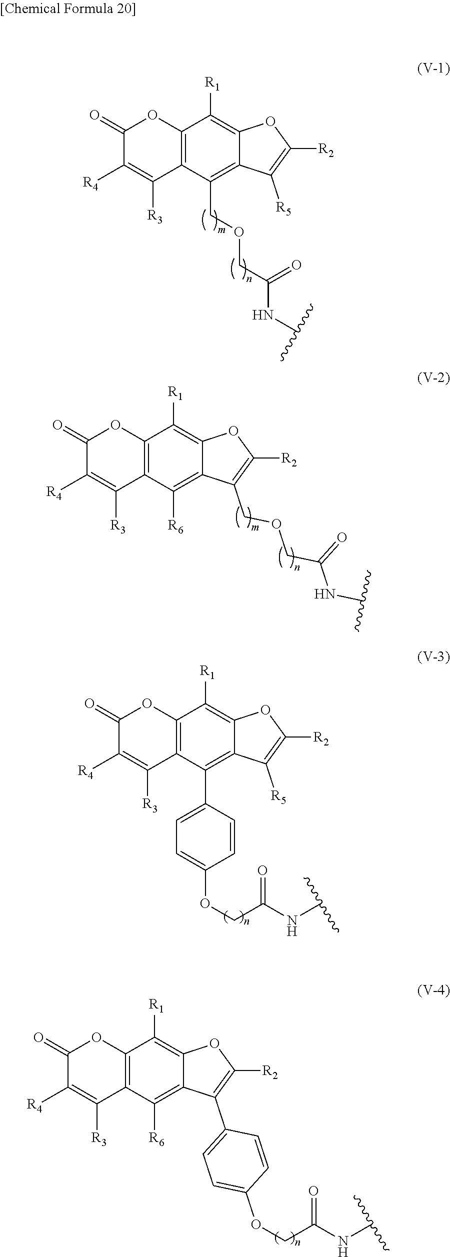

[in the above formulae (V-i), (V-2), (V-3), and (V-4), the definitions of R₁ to R₆ are the same as the definitions of those in the above formula (I), and the definitions of m and n are the same as the definitions of those in the above formulae (III-I) and (III-2)]
(hereinafter, this nucleic acid may be referred to as “the nucleic acid of the present invention”). In the present specification, nucleic acids shall be construed to encompass salts of nucleic acids.

In the formulae (V-1), (V-2), (V-3), and (V-4), the ether bond may be substituted with a thioether bond. However, from the viewpoint of crosslinking efficiency, it is preferable that the ether bond is not substituted with a thioether bond.

In one embodiment, the nucleic acid of the present invention is one in which R₁ is a hydrogen atom, an aliphatic group having 1 to 3 carbon atoms, or —O—R₇ [where R₇ is a hydrogen atom or an aliphatic group having 1 to 3 carbon atoms]. In particular, preferred is one in which R₁ to R₄ in the above formulae (V-1), (V-2), (V-3), and (V-4) are each independently a hydrogen atom or an aliphatic group having 1 to 3 carbon atoms (preferably a methyl group), and more preferred is one in which R₁ to R₄ are all hydrogen atoms or one in which R₁ to R₃ are all methyl groups and R₄ is a hydrogen atom.

In the case where the nucleic acid of the present invention is one in which R₁ is represented by the above formula (II-1), (II-2) or (II-3), R₈ is preferably a hydrogen atom, —N(R₉)₂, or —O—R₉ [where R₉ is a hydrogen atom or an aliphatic group having 1 to 3 carbon atoms (preferably a methyl group)].

In one embodiment, the nucleic acid of the present invention is one in which R₈ or R₆ in the formulae (V-1), (V-2), (V-3), and (V-4) is a hydrogen atom or an aliphatic group having 1 to 3 carbon atoms (preferably a methyl group) and is particularly preferably a hydrogen atom.

In one embodiment, the nucleic acid of the present invention is one in which m in the above formulae (V-1), (V-2), (V-3), and (V-4) is 0 or 1. In another embodiment, the nucleic acid of the present invention is one in which n in the above formulae (V-1), (V-2), (V-3), and (V-4) is 1. Also preferred is one in which m is 0 or 1 and n is 1 in the above formulae (V-1), (V-2), (V-3), and (V-4).

In one embodiment, the nucleic acid of the present invention is a nucleic acid generated through a condensation reaction between the NHS structure of any of the compounds (I-1) to (I-24) and an amino group of the nucleic acid. For example, when the compound is (I-1) (where n is 1) and when the compound is (I-2) (where n is 1), a nucleic acid having a structure represented by the following formula (VI-1) and a nucleic acid having a structure represented by the following formula (VI-2) are obtained, respectively. The same applies to other compounds.

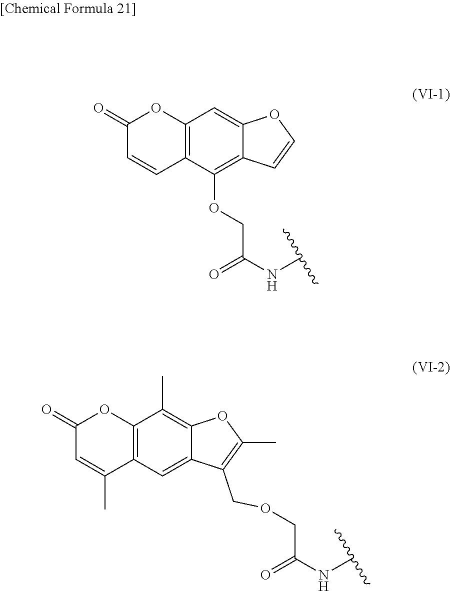 [SMILES: *NC(=O)COc1c2ccoc2cc2oc(=O)ccc12 ; *NC(=O)COCc1c(C)oc2c(C)c3oc(=O)cc(C)c3cc12]

As used herein, the term “nucleic acid” may refer to a monomeric nucleotide, but usually refers to an oligonucleotide composed of a plurality of monomers. Accordingly, unless otherwise stated, when a monomeric nucleotide is intended, it is referred to as a “nucleic acid nucleotide”. Examples of such a nucleic acid include ribonucleic acid (RNA), deoxyribonucleic acid (DNA), peptide nucleic acid (PNA), and morpholino nucleic acid. Further, unless otherwise stated, when a nucleic acid is an oligonucleotide, each of nucleotide residues constituting the nucleic acid (including nucleotides at the 5′ end and the 3′ end) is simply referred to as “nucleotide”. A nucleic acid chain can be produced either fully or partially by chemical synthesis (for example, using an automated synthesizer) or an enzymatic procedure (examples thereof include, but not limited to, use of polymerase, ligase, and a restriction reaction). To the thus-produced nucleic acid chain, an amino linker or the like may be introduced by a method known per se.

The nucleic acid of the present invention can be produced by introducing, into the nucleic acid chain (oligonucleotide) produced by the above-described method, the compound of the present invention using a method known per se. Accordingly, viewed from another aspect, the present invention provides a method for producing the nucleic acid of the present invention, comprising a step of reacting the compound of the present invention with a nucleic acid having an amino group. According to such a method, the nucleic acid of the present invention can be produced by, for example, any of the following steps.

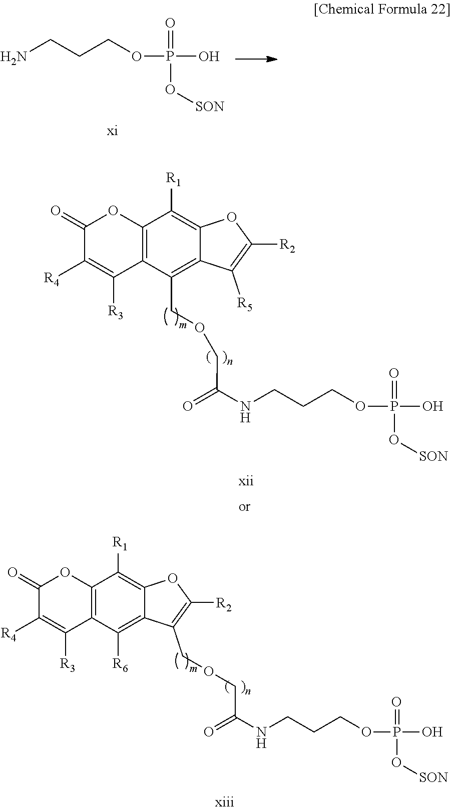

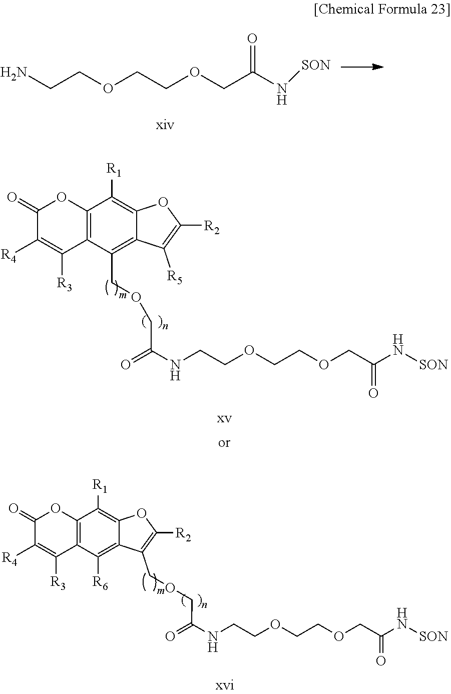

[In the above formulae, the definitions of R₁ to R₆ are the same as the definitions of those in the above formula (I), and the definitions of m and n are the same as the definitions of those in the above formula (III-1). SON represents a single-stranded oligonucleotide.]

Compounds xii, xiii, xv, and xvi can be synthesized by, for example, the following steps. To a sodium carbonate/sodium bicarbonate buffer solution containing the compound xi or xiv is added a DMF solution containing an NHS ester (the compound v or x) at room temperature. The mixture is stirred overnight at room temperature, then the reaction mixture is purified using cytiva NAP™-5 columns and the sample solution is freeze-dried. Further purification is conducted with InertSustainSwift™ C18 5 m 4.6*150 mm column (60° C., solvent A: 0.1 M TEAA aq, solvent B: 0.1 M TEAA 50% ACN aq, gradient solvent B: 0%-100%, 50 min, and flow rate: 0.8 ml/min). The sample is collected and freeze-dried, then dissolved in Milli-Q to give compound xii or xiii.

The nucleic acid of the present invention may be, for example, a triplex-forming oligonucleotide (TFO) or an antisense oligonucleotide (ASO). TFO refers to a nucleic acid having a function of forming a triplex by binding to double-stranded DNA. Bases of the third nucleic acid (TFO) bind to purine bases of purine-pyrimidine base pairs via hydrogen bonds, thereby forming a structure in which three bases are contiguously aligned in a plane to enable triplex formation. Also, a plurality of nucleic acids may be bound via a linker (e.g., a linker obtained by joining one to five 8-amino-3,6-dioxaoctanoic acid linkers) to allow a quadruplex to be formed at some part. As shown in the following structural formulae, there are two types of TFOs: one in which 1′-carbon atoms in bases of TFO face outward (Hoogsteen type binding) (SEQ ID NO: 4) and the other in which 1′-carbon atoms in bases of TFO face the opposite direction (reverse Hoogsteen type binding) (SEQ ID NO: 5). The former TFO exhibits parallel orientation (the same orientation as the strand forming base pairs in the DNA duplex), and the latter TFO exhibits antiparallel orientation (the opposite orientation to the strand forming base pairs in the DNA duplex). The sequences of the respective strands of the DNA duplex (the sequence of the 2^{nd} strand is the target sequence for the TFO, in both the former and latter TFOs) are designated as SEQ ID NO: 6 or 7.

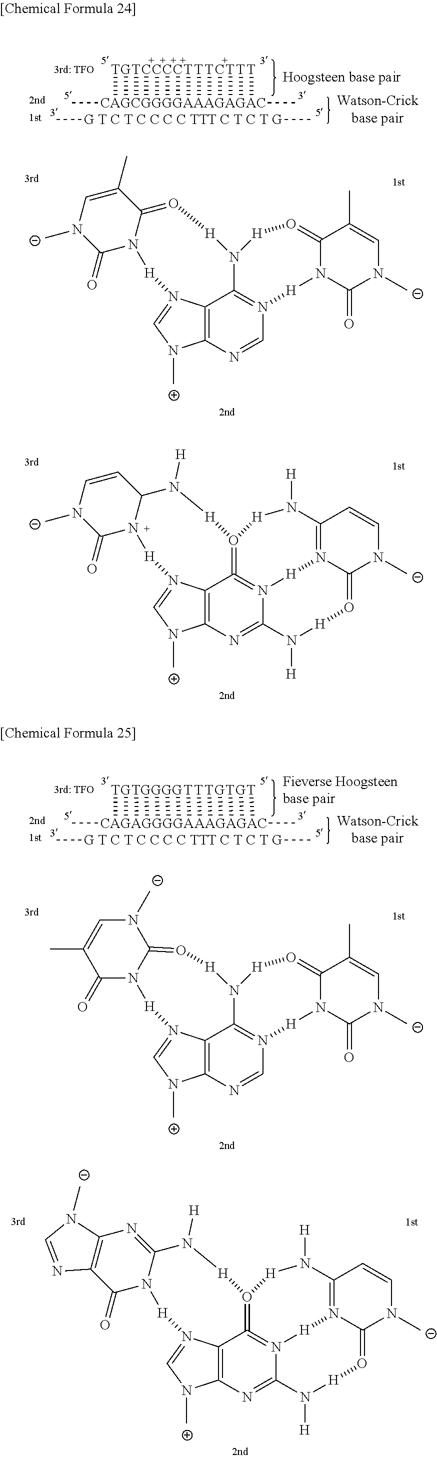

In the TFO exhibiting parallel orientation, to A and G in A-T and G-C base pairs in the DNA duplex bind T (T:A-T base pairs) and protonated C (C⁺:G-C base pairs), respectively. On the other hand, in the TFO exhibiting antiparallel orientation, to A and G in A-T and G-C base pairs in the DNA duplex bind T (T:A-T base pairs) and G (C⁺:G-C base pairs), respectively. Since acidic conditions are required for C to be protonated, the use under physiological conditions is restricted. However, this problem can be avoided by using methylcytidine, which is protonated even under neutral conditions, or by using a derivative that does not require protonation (e.g., 5-methyl-6-oxocytidine).

When the nucleic acid of the present invention is used as TFO, the compound of the present invention can be introduced into the TFO, for example, via an amino group of an amino linker (e.g., an amino linker having 1, 2, 3, 4, 5, or 6 carbon atoms) or a linker (e.g., a PEG linker having an amino group) obtained by substituting at least one or all of the carbon atoms in the above-described amino linker with other atoms (e.g., oxygen atoms), each linker bound to an end of the TFO (e.g., 5′-end, 3′-end, N-end, or C-end) or to a functional group (e.g., a phosphate group) bound to this end, or via an amino group of a base (e.g., adenine, cytosine, or 5-methylcytosine). The thus-obtained TFO forms a triplex with the target double-stranded DNA, and when the triplex is irradiated with light (typically with long-wavelength ultraviolet light (UVA)), crosslink structures are formed between pyrimidine bases of one of the strands of the target double-stranded DNA and pyrimidine bases of the other strand via psoralen included in the TFO.

TFO can be chemically synthesized by, for example, methods known per se, such as those described in International Publication Nos. WO2005/021570, WO03/068695, and WO2001/007455, or methods similar to these methods. Also, it is possible to obtain desired modified oligonucleotides utilizing custom synthesis services.

TFO may be a natural nucleic acid such as DNA or RNA, or a non-natural nucleic acid such as a peptide nucleic acid (PNA). PNA is a molecule that has a structure similar to that of DNA or RNA but has a peptide structure in its main chain. As shown below, in the main chain of PNA, N-(2-aminoethyl)-glycine units, instead of sugars, are linked via amide bonds. Further, purine and pyrimidine rings, which correspond to nucleic acid bases, are linked to the main chain via methylene groups and carbonyl groups.

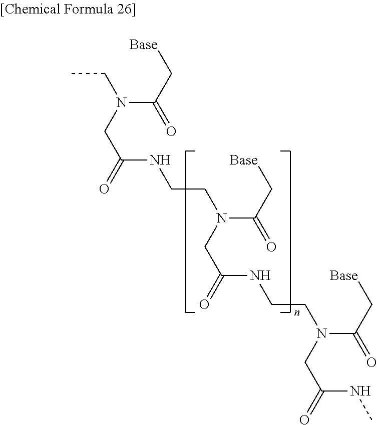

(In the above formula, “Base” represents a natural base or a base with modification (modified base).)

Since charged phosphate moieties as in DNA and RNA are not present in PNA, a PNA/DNA duplex forms stronger bond than a DNA/DNA duplex owing to reduced electrostatic repulsion. Thus, PNA invades the DNA duplex to form a D-loop, thereby forming Watson-Crick base pairs with the complementary DNA strand. Precise molecular recognition also occurs when the PNA strand binds to the complementary DNA, and it is known that a PNA/DNA duplex with mismatched base pairs is more unstable than a DNA/DNA duplex with similar mismatches. Therefore, it is expected that the use of PNA can further reduce off-target effects. In addition, PNA is barely recognizable by nucleases and proteases in vivo, is resistant to degradation by enzymes, and can remain stable over a wide pH range. On the other hand, since charged phosphate moieties are not present in PNA, one or more (e.g., two, three, or four) positively charged molecules (e.g., amino acid residues such as arginine, lysine, and histidine) may be added to the N-terminus, C-terminus, and/or the like of the PNA in order to improve the water solubility of the PNA and/or to enhance interactions with nucleic acids (negatively charged). PNA can be produced using a peptide solid-phase synthesis technique known per se.

As used herein, the term “complementary” means that nucleic acid bases are in a relationship allowing formation of the so-called Watson-Crick base pairs (naturally-occurring base pairs) or non-Watson-Crick base pairs (such as Hoogsteen base pairs or wobble base pairs) via hydrogen bonds. Accordingly, the term “complementary sequence” is intended to encompass not only sequences that are perfectly complementary (i.e., hybridize without mismatches) to the sequence of interest but also sequences with one or more (e.g., two, three, four, five, or more) mismatches as long as they can hybridize to the target sequence under stringent conditions or under physiological conditions of mammalian cells. In other words, the presence of mismatches is acceptable in the complementary strand. For example, the complementary sequence may be a sequence with at least 80% identity (e.g., at least 85%, at least 90%, at least 95%, at least 96%, at least 97%, at least 98%, or at least 99% identity) and most preferably 100% identity with a sequence perfectly complementary to the target sequence.

The stringent conditions may be either low stringent conditions or highly stringent conditions. Low stringent conditions may be conditions at a relatively low temperature and with high salt concentrations, such as, for example, 30° C., 2×SSC, and 0.1% SDS. Highly stringent conditions may be conditions at a relatively high temperatures and with low salt concentrations, such as, for example, 65° C., 0.1×SSC, and 0.1% SDS. The stringency of hybridization can be adjusted by changing the conditions such as the temperature and the salt concentrations. Note here that 1×SSC contains 150 mM sodium chloride and 15 mM sodium citrate.

ASO is a single-stranded oligonucleotide that exhibits an antisense effect (e.g., regulation of gene expression such as reducing the amount of a target RNA or the amount of a protein translated from a target RNA). The target RNA targeted by ASO may be, for example, mRNA, non-coding RNA (ncRNA) (e.g., miRNA), or exogenous RNA (e.g., viral RNA), and is preferably mRNA. As used herein, the term “mRNA” encompasses not only mature mRNA but also mRNA without base modification, unprocessed mRNA precursors (pre-mRNA), and the like. The “target gene” whose expression is regulated by the antisense effect is not limited to a particular gene, and examples thereof include genes derived from an organism into which the complex of the present invention is to be introduced, such as genes that exhibit increased expression in various diseases. As used herein, the term “expression of a gene” is intended to encompass “production of transcription products, such as mRNA and miRNA, encoded by the gene”, but may further encompass “production of a functional protein encoded by a target mRNA”.

Typically, ASO forms a double-stranded region with a target RNA, and the double-stranded region is cleaved by a ribonuclease (e.g., RNase H), resulting in reduction of the amount of the target RNA. Alternatively, an antisense oligonucleotide forms a duplex region with a target RNA to interfere with translation by ribosomes, whereby the expression of a protein encoded by this RNA is inhibited at the translation level. When the nucleic acid of the present invention is used as ASO, the compound of the present invention can be introduced into the ASO by, for example, introducing an amino group into the 5′ end and/or the 3′ end of the ASO, and when the nucleic acid of the present invention is irradiated with light (long-wave ultraviolet light (UV-A)), a crosslinking reaction occurs between psoralen contained in the ASO and uracil on the target RNA, thereby inhibiting the translation of the target RNA into a protein. Accordingly, when the nucleic acid of the present invention is used as ASO, cleavage using a ribonuclease such as RNase H is not considered essential. An amino group can be introduced into the 5′ end or the 3′ end of ASO using a nucleic acid synthesizer, or alternatively, it is possible to obtain such ASO utilizing custom synthesis services.

The target sequence (i.e., the sequence to which ASO hybridizes) in the target RNA is not limited to a particular sequence as long as it is a region present in the RNA, and examples thereof include the 3′ UTR, the 5′ UTR, exons, introns, protein-coding regions, translation initiation region, translation termination regions, and other nucleic acid regions.

Nucleotide molecules constituting the nucleic acid of the present invention may be natural DNA or RNA, may be nucleotide molecules with various types of chemical modification to improve the stability (chemical stability and/or stability towards enzymes) or specific activity (affinity for DNA), or may be nucleic acid analogues. The above-described chemical modification may be, for example, substitution of the phosphate residue (phosphate) of each nucleotide constituting an oligonucleotide with a chemically modified phosphate residue such as phosphorothioate (PS), methylphosphonate, phosphorodithioate, or boranophosphate. Another example of the above-described chemical modification is substitution of the 2′-hydroxy group of the sugar of each nucleotide with another functional group. Examples of the substitution with another functional group include substitution with: a halogen atom (e.g., fluorine atom); a C₁₋₆ alkyl group (e.g., methyl group); an amino group that may be substituted with a C₁₋₆ alkyl group (e.g., methyl group); —OR (where R is, for example, CH₃(2′-O-Me), CH₂CH₂OCH₃ (2′-O-MOE), CH₂CH₂NHC(NH)NH₂, CH₂CONHCH₃, and CH₂CH₂CN).

In addition, the base of the nucleotide may be a modified base, examples of which include, but not limited to, 5-methylcytosine, 5-fluorocytosine, 5-bromocytosine, 5-iodocytosine, N4-methylcytosine, N6-methyladenine, 8-bromoadenine, N2-methylguanine, and 8-bromoguanine. Examples of the nucleic acid analogues include UNA (unlocked nucleic acid), HNA (hexitol nucleic acid), morpholino nucleic acid, and PNA. In the HNA, the hydroxy group of its hexopyranose moiety may be deoxygenated. Further, in the HNA, the hydroxy group of its hexopyranose moiety may be substituted with a fluorine atom.

The chemically modified nucleotide molecule may be a nucleotide having a crosslinked structure (hereinafter also referred to as “bridged nucleic acid (BNA)”). Examples of BNA include locked nucleic acid (LNA) and 2′-O,4′-C-ethylene-bridged nucleic acid (ENA).

Examples of ASO with modification include gapmer oligonucleotides and mixmer oligonucleotides. As used herein, the term “gapmer oligonucleotide” refers to a chimeric antisense oligonucleotide in which an internal region (also referred to herein as “gap region”) having a plurality of (e.g., 5, 6, 7, 8, 9, 10, 11, 12, 13, 14, 15, or more) nucleotides (typically deoxyribonucleotides) recognized by RNase H is located between external regions (the 3′ end external region may be referred to as the “3′ wing region” and the 5′ end external region may be referred to as the “5′ wing region” herein) having at least one (e.g., 1, 2, 3, 4, 5, 6, 7, 8, 9, 10, or more) nucleotide modified so as to confer the resistance against ribonucleases. At least one nucleotide constituting each of the 3′ wing region and the 5′ wing region is preferably a modified nucleotide. Such a modified nucleotide is preferably a bridged nucleic acid. The term “mixmer oligonucleotide” refers to an oligonucleotide in which some (e.g., half) of nucleotides consisting the oligonucleotide are modified nucleotides.

The nucleic acid of the present invention may be modified at the 5′ end, at the 3′ end, and/or inside the sequence of the nucleic acid chain with one or more ligands or fluorophores, and nucleic acids modified with a ligand(s) or fluorophore(s) are also referred to as conjugated nucleic acids. Modifications can be made at the 5′ end, at the 3′ end, and/or inside the sequence by reacting a modifying agent that can react on a solid phase during an elongation reaction on the solid phase. It is also possible to obtain conjugate nucleic acids by synthesizing and purifying nucleic acids to which a functional group such as an amino group, mercapto groups, azide groups, or triple bond has been introduced in advance, and then causing a modifying agent to act thereon. The ligand may be any molecule having affinity for biomolecules, and examples thereof include: lipids such as cholesterol, fatty acids, tocopherol, and retinoids; sugars such as N-acetylgalactosamine (GalNAc), galactose (Gal), and mannose (Man); antibodies such as full antibodies, Fab, and VHH; proteins such as low-density lipoproteins (LDLs) and human serum albumin; peptides such as RGD, NGR, R9, and CPP; low molecular weight substances such as folic acid; synthetic polymers such as synthetic polyamino acids; and nucleic acid aptamers, and they may also be used in any combination. Examples of the fluorophores include those of Cy3 series, those of Alexa series, and Black Hole Quencher.

The length of the nucleic acid of the present invention is not limited to a particular length, and may be at least 7 bases, at least 8 bases, at least 9 bases, at least 10 bases, at least 11 bases, at least 12 bases, or at least 13 bases. Also, the length of the nucleic acid may be 50 bases or less, 45 bases or less, 40 bases or less, 35 bases or less, 30 bases or less, 28 bases or less, 26 bases or less, 24 bases or less, 22 bases or less, 20 bases or less, 18 bases or less, or 16 bases or less.

### 3. Use of Compound or Nucleic Acid of the Present Invention

As described above, the compound and nucleic acid of the present invention intercalate into DNA and crosslink the DNA when irradiated with light. The crosslinked DNA becomes unrepairable, and apoptosis of cells carrying the DNA thus can be induced. Accordingly, the compound and nucleic acid of the present invention can be used suitably for phototherapy. Therefore, viewed from still another aspect, the present invention provides a phototherapeutic agent or phototherapeutic composition containing the compound or nucleic acid of the present invention (hereinafter, they may be referred to as the “agent of the present invention”).

Typical examples of phototherapy include PUVA (psoralen ultra violet A) therapy, and such therapy is usually used for treatment of skin diseases. Examples of such skin diseases include vitiligo vulgaris, psoriasis, parapsoriasis, palmoplantar pustulosis, mycosis fungoides, malignant lymphoma, atopic dermatitis, and alopecia areata. In addition, by using the compound or nucleic acid of the present invention in PUVA therapy performed on cells having a disease gene (e.g., an oncogene or a viral gene), it is also possible to induce apoptosis in a cell-specific manner. Accordingly, the compound and nucleic acid of the present invention are also suitable for treatment of diseases other than skin diseases (e.g., cancers and viral infections).

Examples of cancers that can be treated by the present invention include: cancers such as adenocarcinoma, squamous cell carcinoma, adenosquamous carcinoma, undifferentiated carcinoma, large cell carcinoma, small cell carcinoma, skin cancers (e.g., melanoma and Merkel cell carcinoma), breast cancer, prostate cancer, bladder cancer, vaginal cancer, neck cancer, head and neck cancer, uterus cancer, cervical cancer, liver cancer, kidney cancer, pancreatic cancer, spleen cancer, lung cancer, non-small-cell lung cancer, tracheal cancer, bronchial carcinoma, colon cancer, rectal cancer, small intestine cancer, colorectal cancer, stomach cancer, esophageal cancer, gallbladder cancer, testicular cancer, ovarian cancer, fallopian tube cancer, and nasopharyngeal cancer; cancers in bone tissue, cartilaginous tissue, adipose tissue, muscle tissue, vascular tissue, and hematopoietic tissue; sarcomas such as chondrosarcoma, Ewing sarcoma, rhabdomyosarcoma, malignant hemangioendothelioma, malignant schwannoma, osteosarcoma, and soft tissue sarcoma; blastomas such as hepatoblastoma, medulloblastoma, nephroblastoma, neuroblastoma, pancreatoblastoma, pleuropulmonary blastoma, and retinoblastoma; germ cell tumor; lymphoma; and leukemia, acute myeloid leukemia, and multiple myeloma. Examples of viruses causing viral infections include retroviruses. Specific types of retroviruses will be described below.

The agent of the present invention can be administered to mammals (e.g., humans, rats, mice, guinea pigs, rabbits, sheep, horses, pigs, cows, dogs, cats, and monkeys). Accordingly, the present invention also provides a method for phototherapeutic treatment, or a method for treating skin diseases in a mammal, comprising: administering an effective amount of the compound or nucleic acid of the present invention to a mammal; and irradiating the skin of the mammal with light. The light used in such a method is not limited to particular light as long as it can cause a crosslinking reaction of DNA, and preferably is UVA (with a wavelength of 320 to 400 nm, in particular, 360 to 365 nm). The irradiation time is typically 1 minute or longer (e.g., 5 minutes, 10 minutes, or longer) and 40 minutes or shorter (e.g., 30 minutes, 20 minutes, or shorter). It is also preferable to irradiate one location for 5 to 10 minutes. It is preferable to perform light irradiation after a lapse of a certain period from the administration of the compound or nucleic acid of the present invention, and the period is typically 1 hour or longer (e.g., 2 hours or longer).

When the agent of the present invention contains two or more types of compounds or nucleic acids or further contains other reagents, the agent may be provided in the form of a composition kit comprising the respective compounds or nucleic acids and the reagents in separate compositions.

The agent of the present invention can be administered to a mammal orally or parenterally (e.g., by subcutaneous injection, intramuscular injection, intravenous infusion, local infusion (topical application and local application), intraventricular administration, intrathecal administration, and intraperitoneal administration). The agent for oral administration may be in a solid or liquid dosage form, and specific examples of the dosage form include tablets (including sugar-coated tablets and film-coated tablets), pills, granules, powdered drugs, capsules (including soft capsules), syrups, emulsions, and suspensions. On the other hand, the agent for parenteral administration may be, for example, a topical agent (e.g., an ointment, a cream, or a topical liquid agent), an injection, or a suppository, and the dosage form of the injection may encompass intravenous injections, subcutaneous injections, intradermal injections, intramuscular injections, and intravenous infusions.

As the agent of the present invention, the compound or nucleic acid of the present invention may be used alone in an effective amount or may be formulated together with any desired carrier, for example, a pharmacologically acceptable carrier. Examples of the pharmacologically acceptable carrier include, but not limited to, excipients such as sucrose and starch, binders such as cellulose and methylcellulose, disintegrants such as starch and carboxymethylcellulose, lubricants such as magnesium stearate and Aerosil, aromatic agents such as citric acid and menthol, preservatives such as sodium benzoate and sodium hydrogen sulfite, stabilizers such as citric acid and sodium citrate, suspending agents such as methylcellulose and polyvinyl pyrrolidone, dispersing agents such as surfactants, diluents such as water and saline solution, and base waxes.

When the active ingredient of the agent of the present invention is in the form of a nucleic acid, the agent of the present invention may further contain a reagent for nucleic acid introduction in order to facilitate the introduction of the nucleic acid into target cells. Examples of the reagent for nucleic acid introduction include: calcium chloride, calcium enrichment reagents, and atelocollagen; liposomes; nanoparticles; and cationic lipids such as Lipofectin, Lipofectamine, DOGS (Transfectam), DOPE, DOTAP, DDAB, DHDEAB, HDEAB, polybrene, and poly(ethylenimine) (PEI).

The agent of the present invention may be a pharmaceutical composition in which the nucleic acid of the present invention is encapsulated in liposomes. Liposomes are microscopic closed vesicles having an internal phase enclosed by one or more lipid bilayers, and they are usually capable of holding water-soluble substances in the internal phase and lipid-soluble substances in the lipid bilayer(s). When the term “encapsulated” is used in the present specification, the nucleic acid of the present invention may be held in either the internal phase or the lipid bilayer(s) of the liposomes. The liposomes used in the present invention may be either single-membrane liposomes or multilayer liposomes, and the particle size thereof may be selected as appropriate within a range of, for example, 10 to 1000 nm, preferably 50 to 300 nm. Considering the delivery to target tissue, the particle size is, for example, 200 nm or less and preferably 100 nm or less.

Examples of the method for encapsulating a water-soluble compound such as a nucleic acid in liposomes include the lipid film method (vortex method), reverse phase evaporation method, surfactant removal method, freeze-thaw method, and remote loading method. It should be noted that the method is not limited thereto, and any known method can be selected as appropriate.

The content of the compound or nucleic acid of the present invention in the agent is, for example, about 0.1 to 100 wt % with respect to the total weight of the agent.

The dose of the agent of the present invention varies depending on the purpose of administration, the administration method, the type and severity of the target disease, and the status of a subject to which the agent is to be administered (sex, age, weight, etc.), and for example, when the agent in the form of a nucleic acid is administered systemically to an adult, a single dose of the nucleic acid is usually desirably 2 nmol/kg or more and 50 nmol/kg or less, and when it is administered locally, a single dose of the nucleic acid is usually desirably 1 pmol/kg or more and 10 nmol/kg or less. It is desirable to administer the agent of the present invention in such a dose 1 to 10 times, more preferably 5 to 10 times. The dose may be increased or decreased according to the symptoms.

The agent of the present invention can also be used in combination with other prophylactic or therapeutic medicaments for skin diseases. When used as a concomitant medicament, such a concomitant medicament may be formulated together with the compound or nucleic acid of the present invention and administered in the form of the thus-obtained single formulation, or alternatively, such a concomitant medicament may be formulated separately from the compound or nucleic acid of the present invention (e.g., in the form of a kit) and may be administered simultaneously or in a staggered manner through an administration route that is the same as or different from the administration route of the agent of the present invention. The dose of such a concomitant medicament may be set to an amount normally used when it is administered alone, or may be set smaller than the amount normally used.

The nucleic acid of the present invention is capable of forming a triplex in a target sequence-specific manner, and can crosslink double strands of DNA at or near the triplex-forming region when irradiated with light. The crosslinked region of the DNA is usually removed by a double-strand break (DSB), and the DNA is repaired through non-homologous end-joining (NHEJ). However, owing to errors at this time, insertion, deletion, and the like of nucleotides may occur. Accordingly, the present invention can also be used for genome editing, and viewed from another aspect, the present invention provides a method for producing cells having modified double-stranded DNA (e.g., chromosomal DNA, mitochondrial DNA, and chloroplast DNA; such DNAs are hereinafter collectively referred to as “genomic DNA”), comprising:
(1) a step of contacting double-stranded DNA including a target sequence and present in cells with the nucleic acid of the present invention including a sequence complementary to the target sequence, and
(2) a step of irradiating the cells with light (hereinafter, this method may be referred to as “the method 1 of the present invention”).

Alternatively, by using a donor DNA, DNA repair can occur through homologous recombination (HR) instead of non-homologous end-joining (NHEJ). That is, the nucleic acid of the present invention can also be used for gene knock-in by genome editing. Accordingly, viewed from still another aspect, the present invention provides a method for producing cells having modified double-stranded DNA, comprising:
(1′) a step of brining double-stranded DNA including a target sequence and present in cells, the nucleic acid of the present invention including a sequence complementary to the target sequence, and a donor DNA into contact with each other; and
(2′) a step of irradiating the cells with light (hereinafter, this method may be referred to as “the method 2 of the present invention”).

As used herein, the term “donor DNA” refers to DNA including an exogenous insertion sequence, and the donor DNA usually includes two types of sequences (hereinafter also referred to as “homology arms”) homologous to sequences of two regions (hereinafter also referred to as “adjacent regions”) located upstream and downstream of a target site of double-stranded DNA so as to be adjacent to the target site. The “target site” in the double-stranded DNA refers to a site to be substituted with the insertion sequence included in the donor DNA or a site into which the insertion sequence is to be inserted.

The term “method for producing cells having modified double-stranded DNA” used in the methods 1 and 2 of the present invention should be interpreted as being interchangeable with the term “method for modifying double-stranded DNA present in cells”. As used herein, the term “modification” refers to deletion of a base or base sequence in a target sequence or in the vicinity thereof (e.g., within 100 bases from the 5′ end or the 3′ end of the target sequence) on a DNA strand, substitution of the same with another base or base sequence, and/or insertion of a base or base sequence into a certain region on the DNA strand. The double-stranded DNA to be modified is not limited to particular DNA and is preferably genomic DNA, and modification to the genomic DNA is also referred to as “genome editing”.

In the present specification, a sequence homologous to an adjacent region is not limited to a sequence identical to the sequence of the adjacent region, and may be a sequence preferably with at least 80% identity (e.g., at least 85%, at least 90%, at least 95%, at least 96%, at least 97%, at least 98%, or at least 99% identify) to the identical sequence, provided that homologous recombination can occur in cells.

The insertion sequence may include: a selection marker sequence such as a drug resistance gene (e.g., a kanamycin resistance gene, ampicillin resistance gene, or puromycin resistance gene), thymidine kinase gene, or diphtheria toxin gene; a reporter gene sequence such as a green fluorescent protein (GFP), a red fluorescent protein, P-glucuronidase (GUS), or FLAG; and/or the like, as needed. In addition, in order to allow these genes to be excised after completion of cell selection or the like, the insertion sequence may have LoxP sequences, FRT sequences, or transposon-specific terminal inverted repeat sequences (PiggyBac Terminal Repeats) flanking these genes. A preferable transposon is, for example, piggyBac, which is a transposon derived from a lepidopteran insect (Kaji, K. et al., Nature, 458: 771-775 (2009); Woltjen et al., Nature, 458: 766-770 (2009); WO2010/012077).

Further, as described above, when the nucleic acid of the present invention is used as ASO, it is possible to inhibit translation of the target RNA into a protein. Accordingly, viewed from still another aspect, the present invention provides a method for inhibiting gene expression, comprising:
(I) a step of contacting a target RNA present in cells with the nucleic acid of the present invention including a sequence complementary to a partial sequence (target sequence) of the RNA, and
(II) a step of irradiating the cells with light (hereinafter, this method may be referred to as “the method 3 of the present invention”).

Hereinafter, the term “the method of the present invention” may be used as a collective term for the methods 1 to 3 of the present invention. In the method of the present invention, it is also possible to use a plurality of nucleic acids targeting different sites.

Examples of the cells used in the method of the present invention include bacteria of the genus Escherichia, bacteria of the genus Bacillus, yeasts, insect cells, insects, animal cells, and plant cells.

Examples of the bacteria of the genus Escherichia include Escherichia coli K12 DH1 [Proc. Natl. Acad. Sci. USA, 60, 160 (1968)], Escherichia coli JM103 [Nucleic Acids Research, 9, 309 (1981)], Escherichia coli JA221 [Journal of Molecular Biology, 120, 517 (1978)], Escherichia coli HB101 [Journal of Molecular Biology, 41, 459 (1969)], and Escherichia coli C600 [Genetics, 39, 440 (1954)].

Examples of the bacteria of the genus Bacillus include Bacillus subtilis MI114 [Gene, 24, 255 (1983)] and Bacillus subtilis 207-21 [Journal of Biochemistry, 95, 87 (1984)].

Examples of the yeasts include Saccharomyces cerevisiae AH22, AH22R-, NA87-11A, DKD-5D, and 20B-12, Schizosaccharomyces pombe NCYC1913 and NCYC2036, and Pichia pastoris KM71.

Examples of the insect cells include a cabbage armyworm larva-derived established cell line (Spodoptera frugiperda cells; Sf cells), Trichoplusia ni midgut-derived MG1 cells, Trichoplusia ni egg-derived High Five™ cells, Mamestra brassicae-derived cells, Estigmena acrea-derived cells, and a silkworm-derived established cell line (Bombyx mori N cells; BmN cells). Examples of the Sf cells include Sf9 cells (ATCC CRL1711) and Sf21 cells [both reported in In Vivo, 13, 213-217 (1977)].

Examples of the animal cells include: cells isolated from living organisms; cells in living organisms; cell lines such as monkey COS-7 cells, monkey Vero cells, Chinese hamster ovary (CHO) cells, dhfr-knockout CHO cells, mouse L cells, mouse AtT-20 cells, mouse myeloma cells, rat GH3 cells, HeLa cells, and human FL cells; pluripotent stem cells such as iPS cells and ES cells of humans and other mammals; and primary cultured cells prepared from various tissues. In addition, Zebrafish embryos, Xenopus oocyte, and the like can also be used.

Examples of the plant cells include suspension culture cells, calli, protoplasts, leaf segments, and root segments prepared from various plants (e.g., grain such as rice, wheat, and corn, commercial crops such as tomatoes, cucumbers, and egg plants; garden plants such as carnations and prairie gentians; and experiment plants such as tobacco and Arabidopsis thaliana).

The contact between the double-stranded DNA or target RNA present in the cells and the nucleic acid can be achieved by introducing the nucleic acid into the cells. The nucleic acid can be introduced into the cells according to a known method (e.g., the lysozyme method, competent method, PEG method, CaCl₂ coprecipitation method, electroporation method, microinjection method, particle gun method, lipofection method, or Agrobacterium method) selected depending on the type of the cells. When using a donor DNA, the nucleic acid of the present invention and the donor DNA may be introduced at the same time or at different timings.

The cells into which the nucleic acid has been introduced can be cultured according to a known method selected depending on the type of the cells.

For example, when culturing E. coli or bacteria of the genus Bacillus, medium used for culturing them is preferably liquid medium. Also, the medium preferably contains substances necessary for growing transformants, such as a carbon source, a nitrogen source, and inorganic substances. Examples of the carbon source include glucose, dextrin, soluble starch, and sucrose; examples of the nitrogen source include inorganic and organic substances, such as ammonium salts, nitrates, corn steep liquor, peptone, casein, meat extracts, soybean cake, and potato extracts; and examples of the inorganic substances include calcium chloride, sodium dihydrogenphosphate, and magnesium chloride. Further, yeast extracts, vitamins, growth promoting factors, and the like may be added to the medium. The pH of the medium is preferably about 5 to about 8.

A preferred medium for culturing E. coli is, for example, M9 medium containing glucose and casamino acid [Journal of Experiments in Molecular Genetics, 431-433, Cold Spring Harbor Laboratory, New York 1972]. When necessary, an agent such as 30-indolylacrylic acid may be added to the medium in order to allow promoters to function efficiently. E. coli are usually cultured at about 15° C. to about 43° C. When necessary, aeration or stirring may be performed during culture.

Bacteria of the genus Bacillus are usually cultured at about 30° C. to about 40° C. When necessary, aeration or stirring may be performed during culture.

Examples of medium for culturing yeasts include Burkholder minimum medium [Proc. Natl. Acad. Sci. USA, 77, 4505 (1980)] and SD medium containing 0.5% casamino acid [Proc. Natl. Acad. Sci. USA, 81, 5330 (1984)]. The pH of the medium is preferably about 5 to about 8. The culture temperature is usually about 20° C. to about 35° C. When necessary, aeration or stirring may be performed during culture.

Examples of medium for culturing insect cells or an insect include Grace's Insect Medium [Nature, 195, 788 (1962)] supplemented with additives such as inactivated 10% bovine serum as appropriate. The pH of the medium is preferably about 6.2 to about 6.4. The culture temperature is usually about 27° C. When necessary, aeration or stirring may be performed during culture.

Examples of medium for culturing animal cells include minimum essential medium (MEM) containing about 5% to about 20% fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], and 199 medium [Proceeding of the Society for the Biological Medicine, 73, 1 (1950)]. The pH of the medium is preferably about 6 to about 8. The culture temperature is usually about 30° C. to about 40° C. When necessary, aeration or stirring may be performed during culture.

Examples of medium for culturing plant cells include MS medium, LS medium, and B5 medium. The pH of the medium is preferably about 5 to about 8. The culture temperature is usually about 20° C. to about 30° C. When necessary, aeration or stirring may be performed during culture.

The steps (2), (2′), and (II) of the present invention can be performed, for example, using a commercially available UV-LED irradiator (e.g., OMRON ZUV-C30H). As the light, UVA (with a wavelength of 320 to 400 nm, in particular, 360 to 365 nm) is preferably used. The irradiation time is typically 10 seconds or longer (e.g., 1 minute, 5 minutes, 10 minutes, or longer) and 40 minutes or shorter (e.g., 30 minutes, 20 minutes, or shorter). Typically, it is preferable to start the step (2), (2′), or (II) of the present invention after a lapse of a certain period from the introduction of the nucleic acid of the present invention into the cells, and the period is typically 10 minutes or longer (e.g., 30 minutes, 1 hour, 2 hours, or longer).

The target sequence used in the method of the present invention is not limited to a particular sequence, and examples thereof include sequences of disease-causing factors (e.g., oncogenes and mRNAs encoding such genes, and nucleic acids of viral). When the sequence of a viral nucleic acid is the target sequence, the sequence of the viral nucleic acid may be one that is present independently of the chromosomes of cells, but in one embodiment, it is a sequence derived from a virus incorporated in a chromosome. Such a virus may be, for example, a retrovirus, and examples of the retrovirus include those belonging to the genus Lentivirus, the genus Alpharetrovirus, the genus Betaretrovirus, the genus Gammaretrovirus, and the genus Deltaretrovirus. Examples of the viruses belonging to the genus Lentivirus include human immunodeficiency virus (HIV)-1 and -2, simian immunodeficiency virus (SIV), feline immunodeficiency virus (FIV), and equine infectious anemia virus (EIA). Examples of the viruses belonging to the genus Gammaretrovirus include murine leukemia virus, feline leukemia virus, and reticuloendotheliosis virus. Examples of the viruses belonging to the genus Deltaretrovirus include human T-lymphocytropic virus (HTLV)-I and -II.

Viewed from still another aspect, the present invention also provides an agent for modifying double-stranded DNA and an agent for inhibiting gene expression, each containing the nucleic acid of the present invention. These agents can also be used to treat diseases (e.g., viral infections and cancers). The descriptions regarding the dosage form, administration method (dose, administration route, etc.), administration target, and the like provided above in connection with the agent of the present invention all apply to these agents.

The present invention will be described more specifically with reference to the following examples. It is to be noted, however, that the present invention is not limited to these examples by any means.

### EXAMPLES

### Materials and General Methods

Reagents for the gel electrophoresis were purchased from Wako Pure Chemical Co. 5-Methoxypsoralen was purchased from BIOSYNTH Carbosynth (Kyoto, Japan). All other reagents were purchased from Tokyo Chemical Industry Co. Ltd. (Tokyo, Japan) and used without further purification. Silica gel column chromatography and thin-layer chromatography (TLC) were performed using Silica Gel 60 (spherical) from Kanto Chemical Co., Inc. and Silica Gel 60 F254 from Merck & Co., Inc. Oligodeoxyribonucleotides (ODNs), oligoribonucleotides, and deoxyuridine were synthesized and analyzed by Ajinomoto Genedesign Inc. (Osaka, Japan). NMR spectra were recorded on a JEOL JNM-ECZ400R spectrometer at 400 MHz for ¹H-NMR, 101 MHz for ¹³C-NMR, and 162 MHz for ³¹P-NMR. Chemical shifts were reported in the scale relative to TMS (0.00 ppm for ¹H-NMR, 0.0 ppm for 13C-NMR) or DMSO (2.50 ppm for ¹H-NMR, 39.5 ppm for 13C-NMR) as an internal reference. HR-MS spectrum was taken on a JEOL JMS-700N.

### Synthesis Example 1: Synthesis of succinimidyl-[2-(psoralen-5-yloxy)]-acetate (3)

### Scheme 1, step a: The Synthesis of 5-Hydroxyporalen (7)

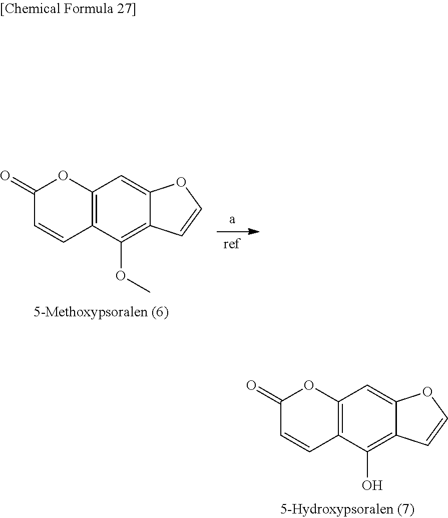 [SMILES: COc1c2ccoc2cc2oc(=O)ccc12 ; O=c1ccc2c(O)c3ccoc3cc2o1]

The synthesis of 5-Hydroxypsoralen was conducted according to the procedure reported in E. C. Row, S. A. Brown, A. V. Stachulski, M. S. Lennard, Org. Biomol. Chem. 2006, 4, 1604-1610.

### Scheme 1, step b: The Synthesis of Compound 8

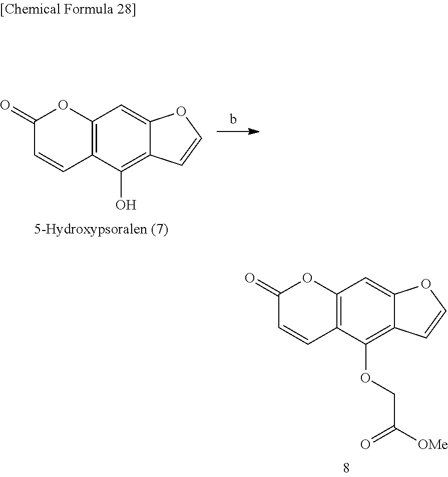 [SMILES: O=c1ccc2c(O)c3ccoc3cc2o1 ; COC(=O)COc1c2ccoc2cc2oc(=O)ccc12]

To a solution of compound 7 (350 mg, 1.731 mmol) and methyl bromoacetate (318 mg, 2.077 mmol) in DMF (4 ml) was added K₂CO₃ (287 mg, 2.077 mmol) at room temperature. The mixture was stirred overnight under an N₂ atmosphere at 80° C., then water was added. The whole was extracted with EtOAc twice. The combined organic layers were dried over Na₂SO₄ and concentrated in vacuo. The residue was dissolved in hexane/EtOAc=3/1 and the resulting precipitate was collected by filtration. The solid was washed with hexane/EtOAc=3/1 and dried in vacuo to give compound 8 (434 mg, 1.583 mmol, 91%) as a white amorphous solid.

### Scheme 1, Step c: The Synthesis of Compound 9

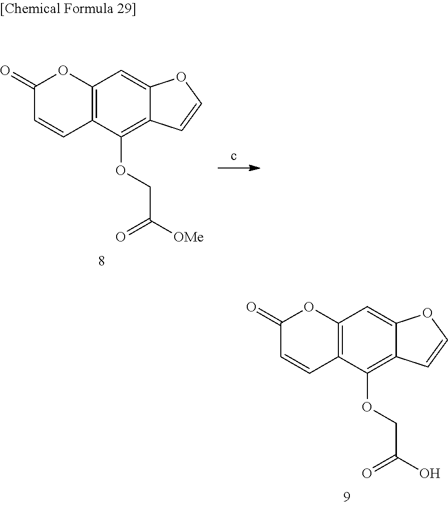 [SMILES: COC(=O)COc1c2ccoc2cc2oc(=O)ccc12 ; O=C(O)COc1c2ccoc2cc2oc(=O)ccc12]

To a solution of compound 8 (434 mg, 1.583 mmol) in MeOH/THF=1/1 (4 ml) was added 1 M NaOH aq (1.6 ml) at room temperature. The mixture was stirred for 1 h, then the mixture was acidified with 5N HCl aq. The whole was extracted with EtOAc five times, and the combined organic layers were dried over Na₂SO₄ and concentrated in vacuo to give compound 9 (375 mg, 1.441 mmol, 91%) as a white amorphous solid.

### Scheme 1, Step d: The Synthesis of Compound 3

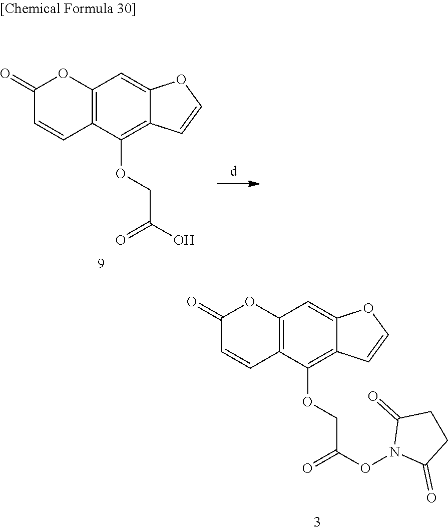 [SMILES: O=C(O)COc1c2ccoc2cc2oc(=O)ccc12 ; O=C(COc1c2ccoc2cc2oc(=O)ccc12)ON1C(=O)CCC1=O]

To a solution of compound 9 (50 mg, 0.192 mmol) and N-hydroxysuccinimide (27 mg, 0.231 mmol) in DMF was added 1-ethyl-3-(3-dimethylaminopropyl)carbodiimide hydrochloride (44 mg, 0.231 mmol) at room temperature. The mixture was stirred overnight at the same temperature under an N₂ atmosphere, then water was added and the whole was extracted with CH₂Cl₂ three times. The combined organic layers were washed with water twice and dried over Na₂SO₄ and concentrated in vacuo to give compound 3 (59 mg, 0.164 mmol, 85%) as a white amorphous solid.

¹H NMR (400 MHz, CDCl₃) δ 2.90 (s, 4H), 5.34 (s, 2H), 6.36 (d, J=9.8 Hz, 1H), 6.94 (dd, J=2.3, 0.9 Hz, 1H), 7.28 (s, 1H), 7.69 (d, J=2.3 Hz, 1H), 8.29 (dd, J=9.8, 0.9 Hz, 1H); ¹³C NMR (101 MHz, (CD₃)₂SO) δ 25.5 (2C), 67.2, 95.0, 104.6, 107.0, 113.3, 113.8, 139.2, 146.8, 147.0, 151.8, 157.3, 160.0, 165.5, 170.0 (2C); FAB-MS (m/z): [M+H⁺] calcd for C₁₇H₁₂NO₈, 358.0563; found 358.0562.

### Synthesis Example 2: Synthesis of (succinimidyl-{2-[(4,5′,8-trimethylpsoralen)-4′-ylmethoxy]}-acetate (4)

### Scheme 2, Step a: The Synthesis of Compound 12

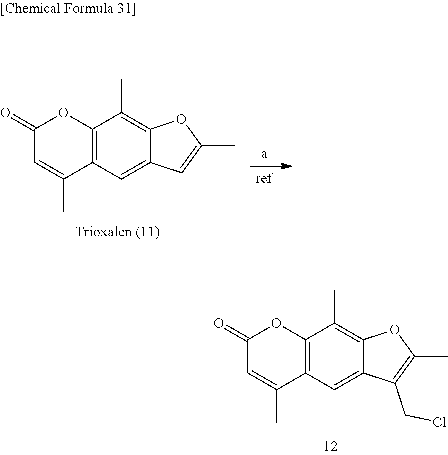 [SMILES: Cc1cc2cc3c(C)cc(=O)oc3c(C)c2o1 ; Cc1oc2c(C)c3oc(=O)cc(C)c3cc2c1CCl]

The synthesis of compound 12 was conducted according to the procedure reported in A. Mukherjee, M. K. Vasquez, Nucleic Acids Res. 2016, 44, 1151-1160.

### Scheme 2, Step b: The Synthesis of Compound 13

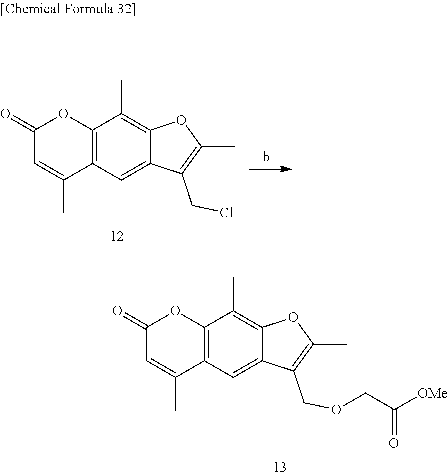 [SMILES: Cc1oc2c(C)c3oc(=O)cc(C)c3cc2c1CCl ; COC(=O)COCc1c(C)oc2c(C)c3oc(=O)cc(C)c3cc12]

A solution of compound 12 (500 mg, 1.807 mmol) in methyl glycolate (5 ml) was heated at 100° C. The mixture was stirred for 5 h at the same temperature under an N₂ atmosphere, then water was added and the whole was extracted with EtOAc three times. The combined organic layers were washed with water twice and dried over Na₂SO₄ and concentrated in vacuo. The residue was purified by silica gel column chromatography (hexane/EtOAc=40:1 to 1:1) to give compound 13 (102 mg, 0.309 mmol, 17%) with some impurities.

### Scheme 2, Step c: The Synthesis of Compound 14

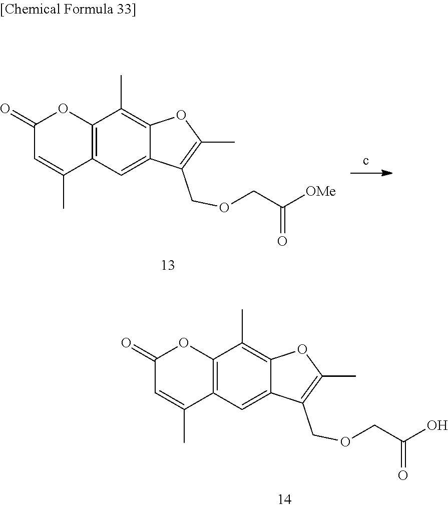 [SMILES: COC(=O)COCc1c(C)oc2c(C)c3oc(=O)cc(C)c3cc12 ; Cc1oc2c(C)c3oc(=O)cc(C)c3cc2c1COCC(=O)O]

To a solution of compound 13 (102 mg, 0.309 mmol) in MeOH/THF=1/1 (4 ml) was added 1 M NaOH aq (309 μl) at room temperature. The mixture was stirred for 3.5 h, then the mixture was acidified with 5N HCl aq. The whole was extracted with EtOAc five times, and the combined organic layers were dried over Na₂SO₄ and concentrated in vacuo to give compound 14 (88 mg, 0.256 mmol, 83%) as a yellow solid.

¹H NMR (400 MHz, CDCl₃) δ 2.48 (s, 3H), 2.49 (s, 3H), 2.56 (s, 3H), 4.09 (s, 2H), 4.74 (s, 2H), 6.23 (s, 1H), 7.70 (s, 1H; ¹³C NMR (101 MHz, (CD₃)₂SO) δ 8.0, 12.0, 18.7, 62.0, 66.4, 107.7, 111.9, 112.2, 112.8, 115.8, 124.9, 148.5, 153.8, 154.0, 155.3, 160.2, 171.9; FAB-MS (m/z): [M+H⁺] calcd for C₁₇H₁₆06, 317.1025, found 317.1024.

### Scheme 2, Step d: The Synthesis of Compound 4

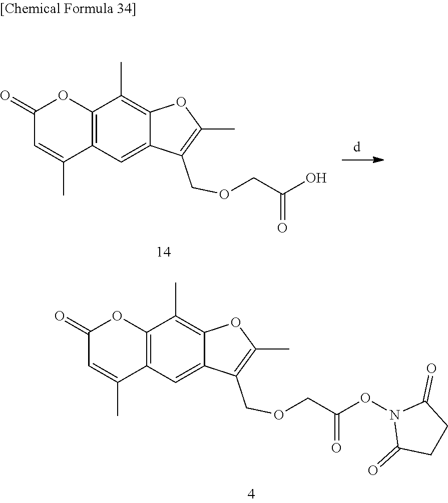 [SMILES: Cc1oc2c(C)c3oc(=O)cc(C)c3cc2c1COCC(=O)O ; Cc1oc2c(C)c3oc(=O)cc(C)c3cc2c1COCC(=O)ON1C(=O)CCC1=O]

To a solution of compound 14 (50 mg, 0.145 mmol) and N-hydroxysuccinimide (20 mg, 0.174 mmol) in DMF (1 ml) was added 1-ethyl-3-(3-dimethylaminopropyl)carbodiimide hydrochloride (33 mg, 0.174 mmol) at room temperature. The mixture was stirred overnight at the same temperature under an N₂ atmosphere, then distilled water was added and the whole was extracted with CH₂Cl₂ three times. The combined organic layers were washed with water twice and dried over Na₂SO₄ and concentrated in vacuo to give compound 4 (53 mg, 0.128 mmol, 88%) as a white amorphous solid.

¹H NMR (400 MHz, CDCl₃) δ 2.51 (s, 3H), 2.52 (s, 3H), 2.58 (s, 3H), 2.89 (s, 4H), 4.44 (s, 2H), 4.83 (s, 2H), 6.25 (s, 1H), 7.68 (s, 1H); ¹³C NMR (101 MHz, (CD₃)₂SO) δ 8.3, 12.0, 18.7, 25.5 (2C), 62.4, 64.6, 107.9, 111.1, 112.3, 112.4, 115.9, 124.6, 148.6, 153.8, 153.9, 156.1, 160.1, 166.7, 170.1 (2C); FAB-MS (m/z): [M+H⁺] calcd for C₂₁H₁₉NO₈SSi, 414.1189, found 414.1188.

### Synthesis Example 3: Post-Functionalization of TFO 15 with an Amino Linker

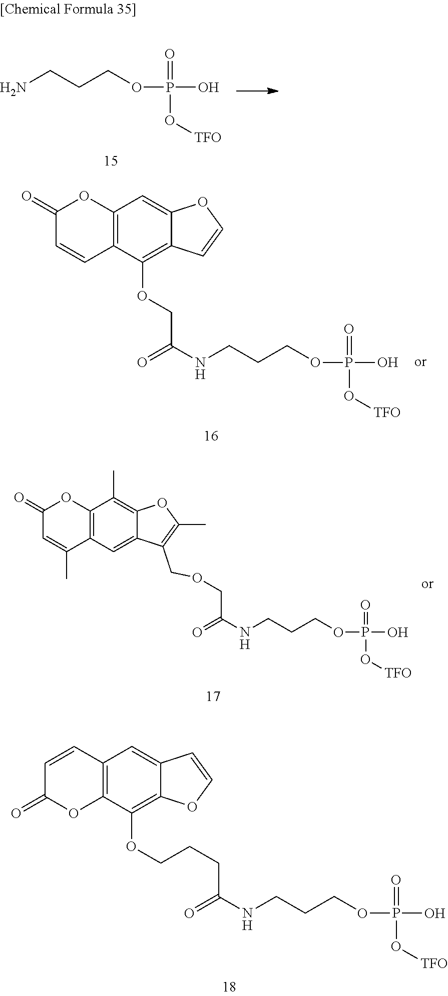

A general procedure for synthesis of Ps-TFOs 16, 17, and 18 (TAMRA labelled oligonucleotides were used for 16 and 17) was as follows. To a sodium carbonate/sodium bicarbonate buffer solution of oligonucleotide 15 (283 M, 50 μl, 14.2 nmol) was added an NHS ester (compound 3 or 4 or SPB) (78 mM, 30 l) in DMF at room temperature. The mixture was stirred overnight at room temperature, then the reaction mixture was purified with cytiva NAP™-5 columns and the sample solution was freeze-dried. Further purification was conducted with Inert Sustain Swift™ C18 5 μm 4.6*150 mm column (60° C., solvent A: 0.1 M TEAA aq, solvent B: 0.1 M TEAA 50% ACN aq, Gradient solvent B: 0-100%, 50 min, flow rate: 0.8 ml/min). The sample was collected (31 min at room temperature) and freeze-dried, then dissolved in Milli-Q to give Ps-TFO 16 (99 μM, 100 μl, 70% yield). Ps-TFO 17 was obtained in 83% yield. Ps-TFO 18 was obtained in 67% yield. The TFOs in this Synthesis Example are DNA.

### Synthesis Example 4: Synthesis of S-Trioxsalen NHS Ester

### Step a: The Synthesis of Compound 19

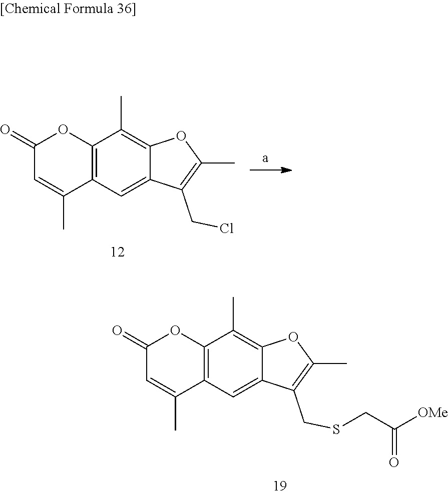 [SMILES: Cc1oc2c(C)c3oc(=O)cc(C)c3cc2c1CCl ; COC(=O)CSCc1c(C)oc2c(C)c3oc(=O)cc(C)c3cc12]

To a solution of compound 12 (200 mg, 723 mol) in DMF (3 ml) was added K₂CO₃ (300 mg, 2.171 mmol) at room temperature. The reaction mixture was heated at 100° C. and stirred for 5 h at the same temperature under an N₂ atmosphere. Then the reaction mixture was concentrated in vacuo and the residue was purified by silica gel column chromatography using CH₂Cl₂ as eluent to give compound 19 (122 mg, 352 μmol, 49%) as a white amorphous solid.

¹H NMR (400 MHz, (CD₃)₂SO) δ 2.46-2.53 (m, 9H), 3.25 (s, 2H), 3.60 (s, 3H), 4.00 (s, 2H), 6.36 (s, 1H), 7.85 (s, 1H); ¹³C NMR (101 MHz, (CD₃)₂SO) δ 8.3, 11.9, 18.8, 24.3, 32.0, 52.1, 107.9, 111.0, 112.3, 112.7, 115.7, 124.5, 148.6, 153.8, 154.0, 154.7, 160.1, 170.6; FAB-MS (m/z): [M+H⁺]C₁₈H₁₉O₅S, 347.0953, found 347.0963.

### Step b: The Synthesis of Compound 20

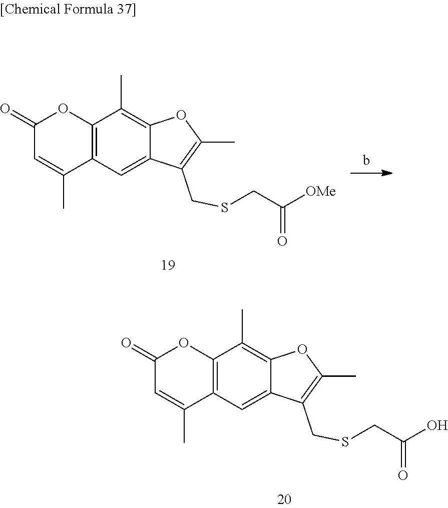 [SMILES: COC(=O)CSCc1c(C)oc2c(C)c3oc(=O)cc(C)c3cc12 ; Cc1oc2c(C)c3oc(=O)cc(C)c3cc2c1CSCC(=O)O]

To a solution of compound 19 (93 mg, 267 mol) in MeOH/THF=1/1 (8 ml) was added 1 M NaOH aq (536 μl) at room temperature. The mixture was stirred for 3.5 h, then the mixture was acidified with 5N HCl aq. The whole was extracted with EtOAc five times, and the combined organic layers were dried over Na₂SO₄ and concentrated in vacuo to give compound 20 (83 mg, 250 mol, 94%) as a pale yellow amorphous solid.

¹H NMR (400 MHz, (CD₃)₂SO) δ 2.46-2.51 (m, 9H), 3.15 (s, 2H), 3.16 (m, 1H), 3.98 (s, 2H), 6.35 (s, 1H), 7.85 (s, 1H); ¹³C NMR (101 MHz, (CD₃)₂SO) δ 8.3, 12.0, 18.8, 24.1, 32.5, 107.9, 111.3, 112.3, 112.7, 115.7, 124.6, 148.6, 153.8, 154.0, 154.5, 160.2, 171.6; FAB-MS (m/z): [M+H⁺] calcd for C₁₇H₁₇O₅S, 333.0797, found 333.0799.

### Step c: The Synthesis of Compound 21

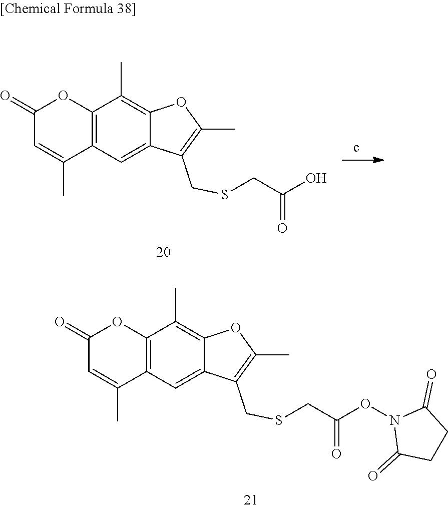 [SMILES: Cc1oc2c(C)c3oc(=O)cc(C)c3cc2c1CSCC(=O)O ; Cc1oc2c(C)c3oc(=O)cc(C)c3cc2c1CSCC(=O)ON1C(=O)CCC1=O]

To a solution of compound 20 (54 mg, 163 mol) and N-hydroxysuccinimide (23 mg, 196 mol) in DMF (1 ml) was added 1-ethyl-3-(3-dimethylaminopropyl)carbodiimide hydrochloride (38 mg, 196 mol) at room temperature. The mixture was stirred overnight at the same temperature under an N₂ atmosphere, then distilled water was added and the whole was extracted with CH₂Cl₂ three times. The combined organic layers were washed with water twice and dried over Na₂SO₄ and concentrated in vacuo to give compound 21 (65 mg, 151 mol, 93%) as a yellow amorphous solid.

¹H NMR (400 MHz, (CD₃)₂SO) δ 2.46 (s, 3H), 2.47 (s, 3H), 2.48 (s, 3H), 2.86 (s, 4H), 3.70 (s, 2H), 4.11 (s, 2H), 6.35 (s, 1H), 7.86 (s, 1H); ¹³C NMR (101 MHz, (CD₃)₂SO) δ 8.3, 11.9, 18.8, 24.4, 25.6 (2C), 29.4, 108.0, 110.3, 112.3, 112.6, 115.8, 124.3, 148.6, 153.8, 154.0, 155.0, 160.1, 166.6, 170.2 (2C); FAB-MS (m/z): [M+H⁺] calcd for C₂₁H₂₀NO₇S, 430.0960, found 430.0959.

### Synthesis Example 5: Post-Functionalization of TFO 22 with a PEG Linker Having an Amino Group

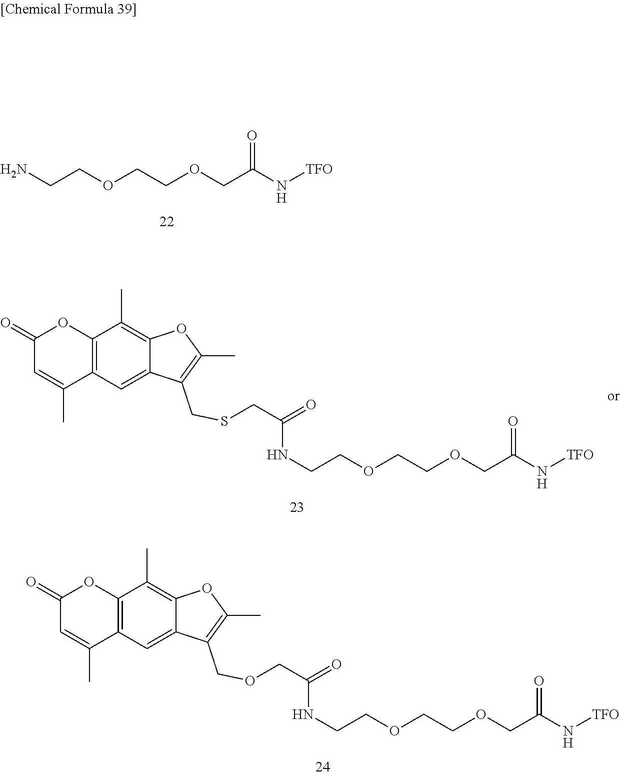

A general procedure for synthesis of Ps-TFOs 23 and 24 was as follows. To a sodium carbonate/sodium bicarbonate buffer solution of oligonucleotide 22 (50 μM, 50 μl, 2.5 nmol) was added an NHS ester (compound 4 or 21) (2.5 mM, 5 μl) in DMF at room temperature. The mixture was stirred (at 1,500 rpm) overnight at room temperature, then the reaction mixture was purified with cytiva NAP™-5 columns and the sample solution was freeze-dried. Further purification was conducted with InertSustainSwift™ C18 5 μm 4.6*150 mm column (60° C., solvent A: 0.1% TFA (trifluoroacetic acid) in 10% MeCN (acetonitrile), solvent B: 0.1% TFA in MeCN, Gradient solvent B: 0-100%, 50 min, flow rate: 0.8 ml/min). The sample was collected (14 min at room temperature) and freeze-dried to give Ps-TFO 23 (34% yield) or Ps-TFO 24 (52% yield). The TFOs in this Synthesis Example are PNAs.

### Example 1: Photo-Crosslinking Reaction and Gel-Shift Assay

Sample solutions were prepared in 10 mM phosphate buffer (pH 5.3) containing 200 mM NaCl, 100 M EDTA and the oligonucleotide. The concentration was set at 1.0 μM. The samples were heated at 95° C. for 3 min and slowly cooled from 95° C. to 4° C. at 0.5° C./min for annealing. The samples were then UV-irradiated for 0-30 sec using a UV-LED irradiator (OMRON ZUV-C30H, 365 nm), followed by analysis using denaturing PAGE (15% polyacrylamide/7 M urea/25% formamide). The crosslinking efficiencies were quantified by measuring TAMRA fluorescence.

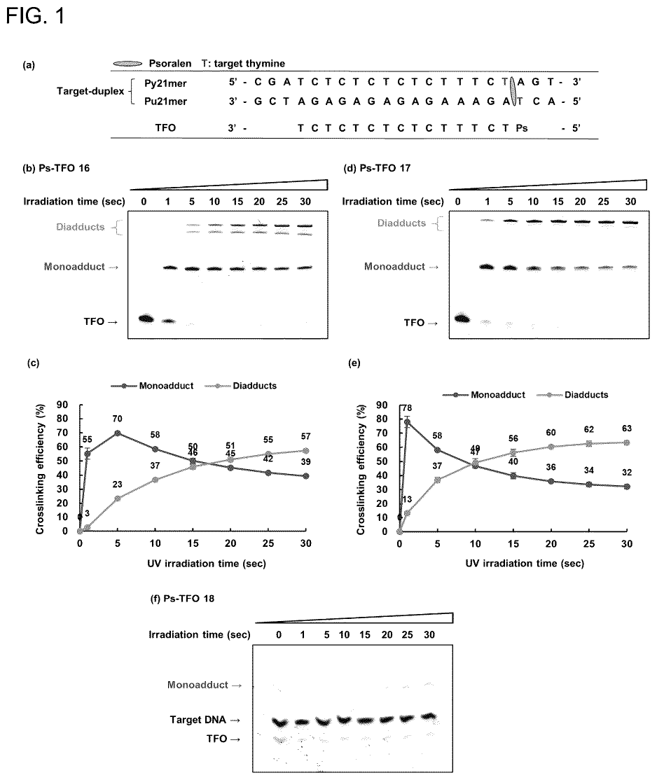

The results are shown in FIG. 1. As can be seen from FIG. 1, with Ps-TFOs 16 and 17, the number of diadducts forming triple-stranded nucleic acids increased in proportion to the UV irradiation time. In contrast, with Ps-TFO 18, the formation of diadducts was not observed, and besides, even monoadducts formed between the Ps-TFO and a single strand of the nucleic acid were almost not detectable.

### Example 2: Comparison of Crosslinking Efficiency Between S-Linker Trioxsalen-PNA and O-Linker Trioxsalen-PNA

The crosslinking efficiencies of S-linker Trioxsalen-PNA and O-linker Trioxsalen-PNA were evaluated under the same conditions as in Example 1. The crosslinking efficiencies were quantified by measuring TAMRA fluorescence.

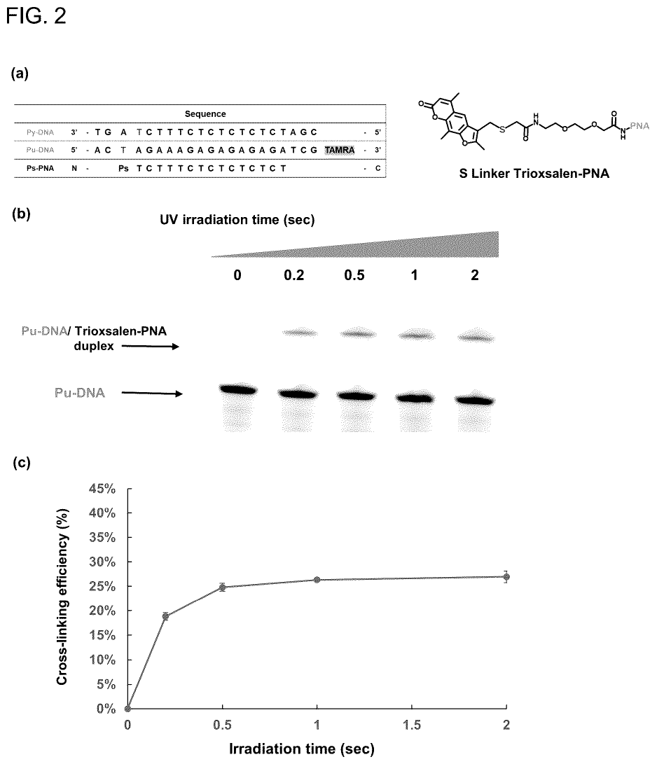

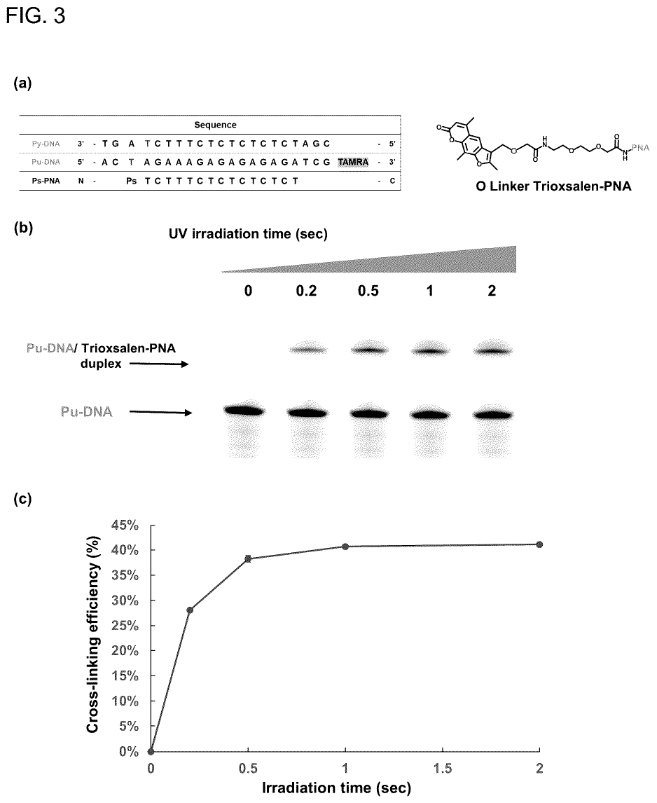

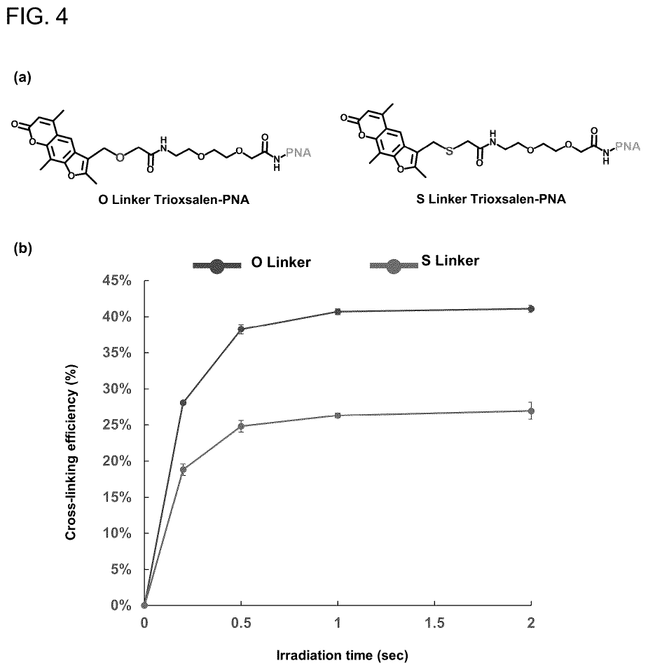

The results are shown in FIG. 2 (S-linker Trioxsalen-PNA) and FIG. 3 (O-linker Trioxsalen-PNA). Also, FIG. 4 shows a graph comparing the crosslinking efficiencies of S-linker Trioxsalen-PNA and O-linker Trioxsalen-PNA.

The above results show that, although S-linker Trioxsalen-PNA formed diadducts with the target ds-DNA as with O-linker Trioxsalen-PNA, both the yield and the crosslinking efficiency achieved by 0-linker Trioxsalen-PNA were higher than those achieved by S linker Trioxsalen-PNA. In particular, the crosslinking efficiency of the O linker Trioxsalen-PNA was markedly higher than that of S linker Trioxsalen-PNA.

### Example 3: Evaluation of Intracellular Activity of Trioxsalen-Tc-PNA

293FT cells were seeded in a 96-well plate at a density of 1.5×10⁴ cells/well and incubated at 37° C. for 40 hours. Subsequently, the supernatant was removed from the cell-seeded wells, and 90 μL of medium was added. Samples were added to the medium {lipofectamine 2000+Opti-MEM+ (annealed and UV-irradiated sample: plasmid+Tri-tc-PNA)}, and the cells were incubated at 37° C. for 24 hours. The medium was then replaced, and the cells were incubated at 37° C. for 32 hours. The intracellular activity of Tri-tc-PNA was evaluated by Luciferase assay.

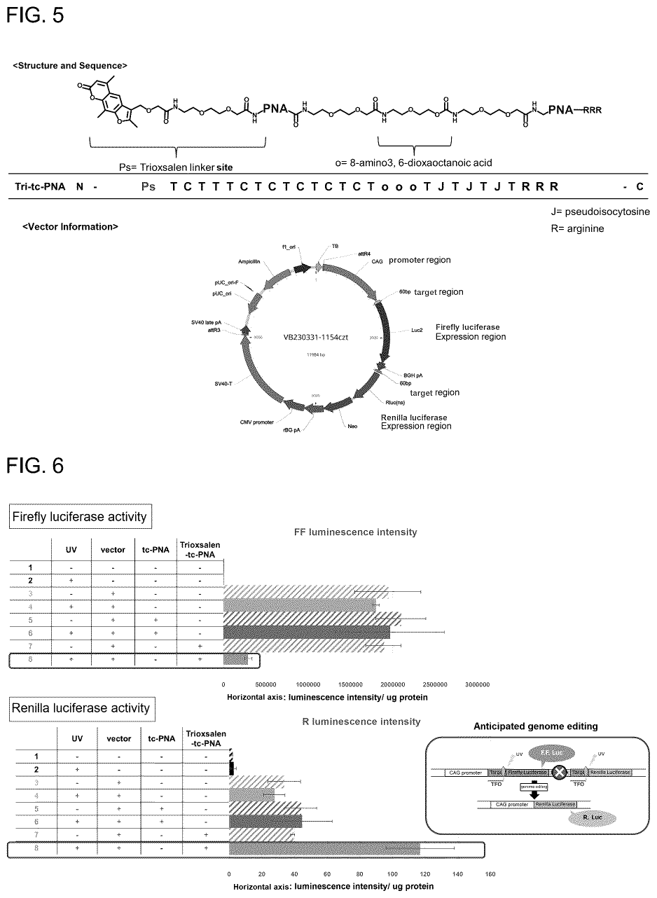

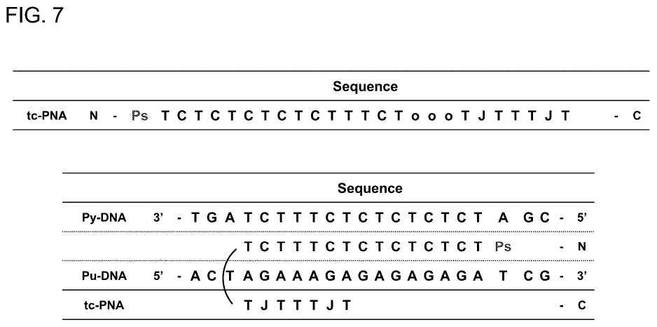

The results are shown in FIG. 6. As can be seen from FIG. 6, the highest Renilla luciferase activity was observed when the plasmid vector containing Trioxsalen-tc-PNA and the target sequence was introduced into the cells and irradiated with UV light. As can be seen from the sequence shown in FIG. 5, there are two mismatched base pairs between Trioxsalen-tc-PNA and the target sequence. Nevertheless, Trioxsalen-tc-PNA was found to exhibit intracellular activity. This demonstrates that, although the nucleic acid of the present invention preferably has a sequence that perfectly matches the target sequence, a certain number of mismatches are acceptable.

### Example 4: Evaluation of Apoptosis-Inducing Activity of Trioxsalen-PNA

HeLa cells (HPV18 positive) were seeded in a 96-well plate at a density of 5000 cells/well and incubated for 24 hours. Next, a psoralen-introduced nucleic acid (targeting hPV18 mRNA) was introduced into the cells (Lipofectamine 2000, the nucleic acid concentration was adjusted to around 100 nM to 500 nM), and the cells were incubated for 6 hours. Thereafter, the cells were UV-irradiated (2 mW/cm², 10 min). After incubating the cells for 48 hours, the number of viable cells was measured (WST-8 assay).

### INDUSTRIAL APPLICABILITY

The present invention provides novel psoralen compounds that have high DNA crosslinking efficiency and can be introduced into nucleic acids under standard labeling conditions using NHS esters. The psoralen compounds are useful for applications such as phototherapeutic agents and genome editing.

The present application claims priority based on Japanese Patent Application Nos. 2022-206081 (filed in Japan on Dec. 22, 2022) and 2023-106429 (filed in Japan on Jun. 28, 2023), the disclosures of which are incorporated herein in their entirety by reference.

1. A compound represented by the following formula (I) or a salt thereof: 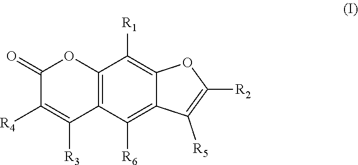
wherein
R₁ is a hydrogen atom, an aliphatic group having 1 to 3 carbon atoms, or —O—R₇ [where R₇ is a hydrogen atom or an aliphatic group having 1 to 3 carbon atoms], or is represented by the following formula (II-1), (II-2), or (II-3): 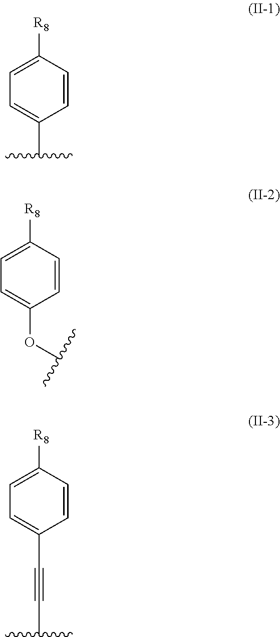
[in the formulae (II-1), (II-2), and (II-3), R₈ is a hydrogen atom, an aliphatic group having 1 to 3 carbon atoms, or —N(R₉)₂ or —O—R₉ [where R₉ is a hydrogen atom or an aliphatic group having 1 to 3 carbon atoms]],
R₂ is a hydrogen atom, an aliphatic group having 1 to 3 carbon atoms, or —O—R₁₀ [where R₁₀ is a hydrogen atom or an aliphatic group having 1 to 3 carbon atoms],
R₃ and R₄ are each independently a hydrogen atom, an aliphatic group having 1 to 3 carbon atoms, —O—R₁₁ [where R₁₁ is a hydrogen atom or an aliphatic group having 1 to 3 carbon atoms], or a phenyl group or naphthyl group in which each hydrogen atom may be substituted with an aliphatic group having 1 to 3 carbon atoms, or alternatively,
R₃ and R₄ together form a benzene ring or naphthyl ring in which each hydrogen atom may be substituted with an aliphatic group having 1 to 3 carbon atoms,
R₅ is represented by the following Formula (III-1) or (III-2): 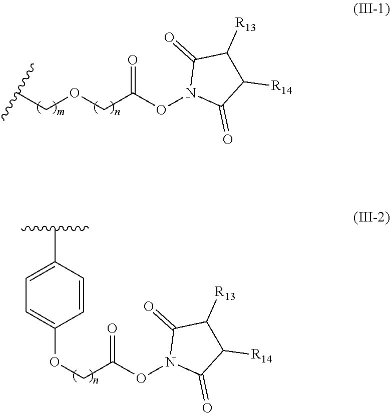
[in the formulae (III-1) and (III-2),
m is an integer of 0 to 4,
n is an integer of 1 to 5, and
R₁₃ and R₁₄ are each independently a hydrogen atom, an aliphatic group having 1 to 3 carbon atoms, or a polar group], and
R₆ is a hydrogen atom, an aliphatic group having 1 to 3 carbon atoms, or —O—R₁₂ [where R₁₂ is a hydrogen atom or an aliphatic group having 1 to 3 carbon atoms].

2. The compound or salt thereof according to claim 1, wherein R₁ to R₄ are each independently a hydrogen atom or a methyl group.

3. The compound or salt thereof according to claim 1, wherein R₁₃ is a sulfo group.

4. The compound or salt thereof according to claim 1, wherein mis 0 or 1.

5. (canceled)

6. The compound according to claim 1, represented by the following formula (IV-2):

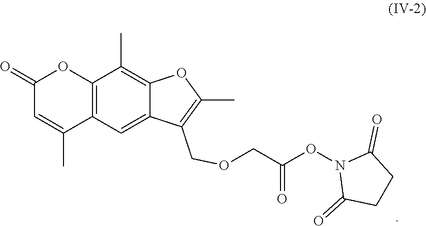 [SMILES: Cc1oc2c(C)c3oc(=O)cc(C)c3cc2c1COCC(=O)ON1C(=O)CCC1=O]

7. A nucleic acid having a structure represented by the following formula (V-1), (V-2), (V-3), or (V-4): 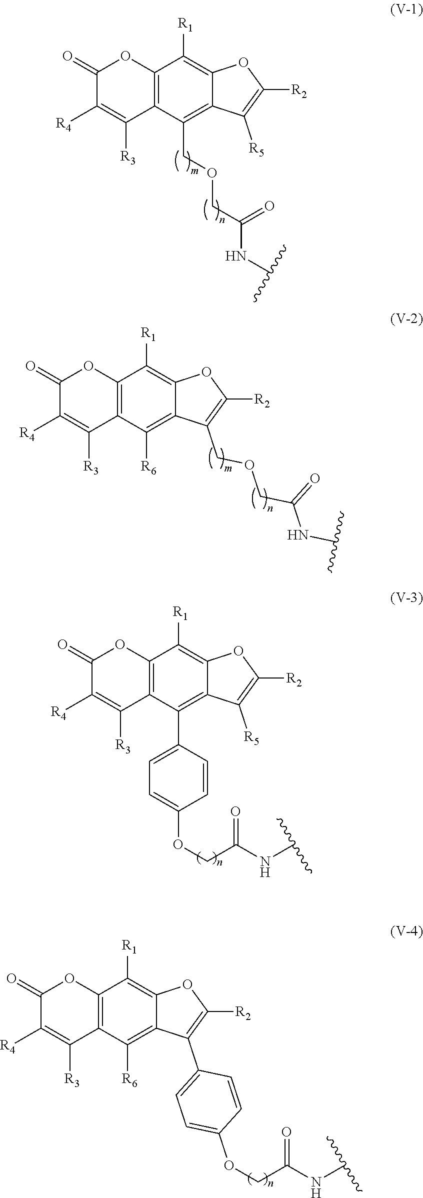
[in the formulae (V-1), (V-2), (V-3), and (V-4),
R₁ is independently a hydrogen atom, an aliphatic group having 1 to 3 carbon atoms, or —O—R₇ [where R₇ is a hydrogen atom or an aliphatic group having 1 to 3 carbon atoms], or is represented by the following formula (II-1), (II-2), or (II-3): 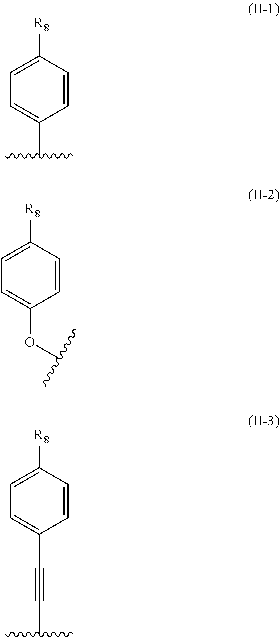
[in the formulae (II-1), (II-2), and (II-3), R₈ is a hydrogen atom, an aliphatic group having 1 to 3 carbon atoms, or —N(R₉)₂ or —O—R₉ [where R₉ is a hydrogen atom or an aliphatic group having 1 to 3 carbon atoms]],
R₂ is a hydrogen atom, an aliphatic group having 1 to 3 carbon atoms, or —O—R₁₀ [where R₁₀ is a hydrogen atom or an aliphatic group having 1 to 3 carbon atoms],
R₃ and R₄ are each independently a hydrogen atom, an aliphatic group having 1 to 3 carbon atoms, —O—Ru [where R₁₁ is a hydrogen atom or an aliphatic group having 1 to 3 carbon atoms], or a phenyl group or naphthyl group in which each hydrogen atom may be substituted with an aliphatic group having 1 to 3 carbon atoms, or alternatively,
R₃ and R₄ together form a benzene ring or naphthyl ring in which each hydrogen atom may be substituted with an aliphatic group having 1 to 3 carbon atoms,
R₅ and R₆ are each independently a hydrogen atom, an aliphatic group having 1 to 3 carbon atoms, or —O—R₁₂ [where R₁₂ is a hydrogen atom or an aliphatic group having 1 to 3 carbon atoms],
m is an integer of 0 to 4, and
n is an integer of 1 to 5].

8. The nucleic acid according to claim 7, wherein R₁ to R₄ are each independently a hydrogen atom or a methyl group.

9. The nucleic acid according to claim 7, having a structure represented by the following formula (VI-1) or (VI-2):

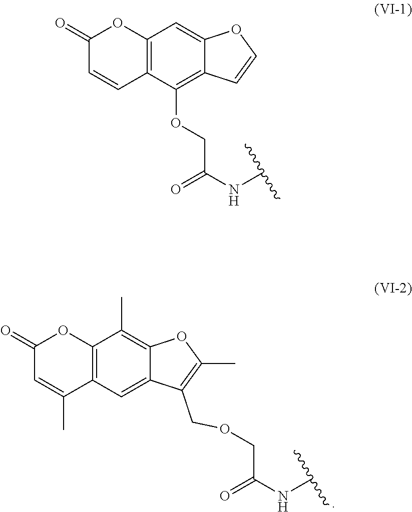 [SMILES: *NC(=O)COc1c2ccoc2cc2oc(=O)ccc12 ; *NC(=O)COCc1c(C)oc2c(C)c3oc(=O)cc(C)c3cc12]

10. The nucleic acid according to claim 7, which is a triplex-forming oligonucleotide or an antisense oligonucleotide.

11. The nucleic acid according to claim 7, wherein the triplex-forming oligonucleotide is DNA or PNA.

12. A method for producing the nucleic acid according to claim 7, comprising a step of reacting a compound represented by the following formula (I) or a salt thereof. 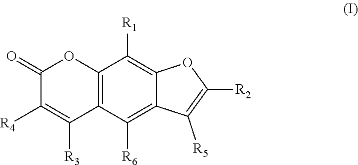
[in the formula (I):
R₁ is a hydrogen atom, an aliphatic group having 1 to 3 carbon atoms, or —O—R₇ [where R₇ is a hydrogen atom or an aliphatic group having 1 to 3 carbon atoms], or is represented by the following formula (II-1), (II-2), or (II-3): 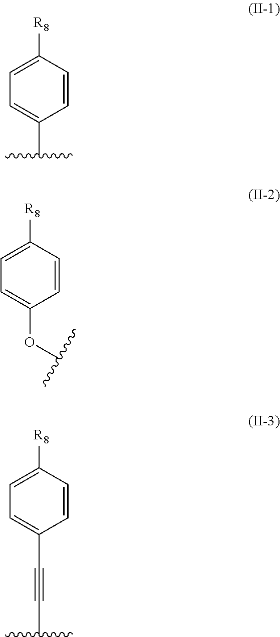
[in the formulae (II-1), (II-2), and (II-3), R₈ is a hydrogen atom, an aliphatic group having 1 to 3 carbon atoms, or —N(R₉)₂ or —O—R₉ [where R₉ is a hydrogen atom or an aliphatic group having 1 to 3 carbon atoms]],
R₂ is a hydrogen atom, an aliphatic group having 1 to 3 carbon atoms, or —O—R₁₀ [where R₁₀ is a hydrogen atom or an aliphatic group having 1 to 3 carbon atoms],
R₃ and R₄ are each independently a hydrogen atom, an aliphatic group having 1 to 3 carbon atoms, —O—R₁₁ [where R₁₁ is a hydrogen atom or an aliphatic group having 1 to 3 carbon atoms], or a phenyl group or naphthyl group in which each hydrogen atom may be substituted with an aliphatic group having 1 to 3 carbon atoms, or alternatively,
R₃ and R₄ together form a benzene ring or naphthyl ring in which each hydrogen atom may be substituted with an aliphatic group having 1 to 3 carbon atoms, and
one of R₅ and R₆ is a hydrogen atom, an aliphatic group having 1 to 3 carbon atoms, or —O—R₁₂ [where R₁₂ is a hydrogen atom or an aliphatic group having 1 to 3 carbon atoms] and the other one of R₅ and R₆ is represented by the following Formula (III-1) or (III-2): 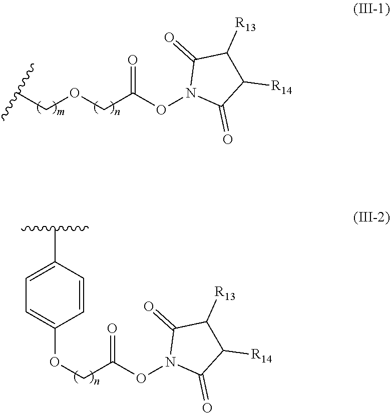
[in the formulae (III-1) and (III-2),
m is an integer of 0 to 4,
n is an integer of 1 to 5, and
R₁₃ and R₁₄ are each independently a hydrogen atom, an aliphatic group having 1 to 3 carbon atoms, or a polar group]]
with a nucleic acid having an amino group.

13. A method for phototherapeutic treatment of a mammal, comprising a step of administering an effective amount of the nucleic acid according to claim 7 to the mammal, and a step of irradiating the skin of the mammal with light.

14. A method for producing cells having modified double-stranded DNA, comprising
(1) a step of contacting double-stranded DNA including a target sequence and present in cells with the nucleic acid according to claim 7 including a sequence complementary to the target sequence, and
(2) a step of irradiating the cells with light.

15. The method according to claim 14, wherein the target sequence is derived from a virus.

16. The nucleic acid according to claim 7, having a structure represented by the following formula (VI-1):

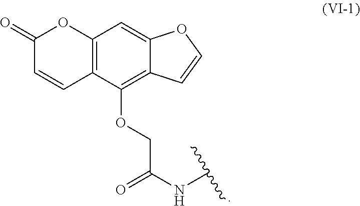 [SMILES: *NC(=O)COc1c2ccoc2cc2oc(=O)ccc12]

17. The nucleic acid according to claim 7, having a structure represented by the following formula (VI-2): 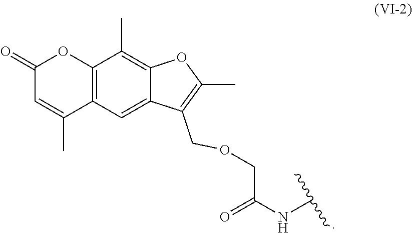 [SMILES: *NC(=O)COCc1c(C)oc2c(C)c3oc(=O)cc(C)c3cc12]
